(19) United States
(12) Patent Application Publication
(10) Pub. No.: US 2022/0290214 A1
(43) Pub. Date: Sep. 15, 2022

(54) PRIMER SET FOR DETECTING FOOD POISONING BACTERIA BY USING NEXT-GENERATION SEQUENCING METHOD AND METHOD FOR DETECTING FOOD POISONING BACTERIA BY USING THE PRIMER SET

(71) Applicants: Sanigen Co., Ltd., Anyang-si (KR); Korean Ministry of Food and Drug Safety, Cheongju-si (KR)

(72) Inventors: Jeongwoong Park, Yongin-si (KR); Jinho Choi, Seoul (KR); Eunsu Ha, Seoul (KR); Byungcheol Kang, Gunpo-si (KR); Iseul Choi, Anyang-si (KR); Joseph Lee, Seoul (KR); Hongjun Na, Seoul (KR); Hyejin Moon, Uiwang-si (KR); Soon Han Kim, Cheongju-si (KR); Seung Hwan Kim, Cheongju-si (KR); Woo Jung Lee, Cheongju-si (KR)

(21) Appl. No.: 17/692,236

(22) Filed: Mar. 11, 2022

(30) Foreign Application Priority Data
Mar. 11, 2021 (KR) 10-2021-0032317
Mar. 10, 2022 (KR) 10-2022-0029865

(51) Int. Cl. C12Q 1/689; C12N 15/10

(52) U.S. Cl. CPC C12Q 1/689; C12N 15/1086; C12Q 2600/16; C12R 2001/085

(57) ABSTRACT

The present invention relates to a primer set for detecting food poisoning bacteria by using a next-generation sequencing method and a method for detecting food poisoning bacteria by using the primer set. Specifically, with the primer set in accordance with the present invention, 16 kinds of food poisoning bacteria can be detected at one time, for a shorter time, and with high accuracy, and the primer set can be useful for simultaneous detection of food poisoning bacteria.

### CROSS-REFERENCE TO RELATED APPLICATIONS

The present application claims priority to Korean Application No. 10-2021-0032317 filed on Mar. 11, 2021 and Korean Application No. 10-2022-0029865 filed on Mar. 10, 2022, which applications are incorporated herein by reference.

### SEQUENCE LISTING

The present application contains a Sequence Listing which has been submitted electronically in ASCII format and is hereby incorporated by reference in its entirety. The ASCII copy, created on Apr. 7, 2022, is named SAN-P30001 ST25.txt and is 15,655 bytes in size.

### TECHNICAL FIELD

The present invention relates to a primer set for detecting food poisoning bacteria by using a next-generation sequencing method and a method for detecting food poisoning bacteria by using the primer set.

### BACKGROUND ART

Food poisoning refers to diseases associated with infection or toxin caused by consumption of food or water, and it can cause symptoms such as vomiting, diarrhea, abdominal pain, and fever, and can cause, in severe cases, death. As the number of food poisoning outbreaks continues to increase with the development of group meals, food industry, and ready-to-eat food continue to increase, and as the import and export of food increases internationally, it is necessary to quickly and economically identify food poisoning in order to meet consumer demands for food safety or meet international distribution regulations.

Food poisoning is caused by bacteria, viruses, chemicals, parasites, and so on, and food can be contaminated in various ways throughout the entire flow from food production to cultivation, packaging, distribution, and consumption. Food poisoning bacteria that cause food poisoning are generally identified by biochemical or immunological methods after incubating a sample in a selective medium and separating the bacteria. An immunological method can detect bacteria with high accuracy, but it costs a lot because it requires a large amount of sample and requires different antibodies for different bacteria. In addition, it is difficult to store and use antibodies, only one type of or a limited number of bacteria can be detected at a time by using an antibody, and detection time is long.

In recent years, polymerase chain reaction (PCR) has begun to be used for detecting food poisoning bacteria, and the demand is increasing due to its high accuracy, simplicity and quickness. In particular, a real-time PCR method has been obtaining high popularity because it has advantageous, for example, in that electrophoresis does not need to be performed since a PCR amplification product can be detected in real time, and in that not only detection accuracy and sensitivity are excellent but also detection reproducibility is high, detection can be automated, and a quantified detection result can be obtained. In addition, as a probe labeled with a fluorescent marker is used, less amount of a sample is needed than when detection is performed by using a DNA chip or an antigen-antibody reaction method.

Korean Patent No. 10-1456646, which is directed to a kit for detecting food poisoning bacteria and a method for detecting food poisoning bacteria by using the kit, discloses a kit for detecting food poisoning bacteria that can efficiently detect various types of food poisoning bacteria at the same time.

### SUMMARY OF DISCLOSURE

An object of the present invention is to provide a primer set for detecting food poisoning bacteria.

Another object of the present invention is to provide a composition comprising the primer set and a method of using the primer set.

In order to achieve the above objects, the present invention provides a primer set for detecting food poisoning bacteria, the primer set comprising at least one selected from the group consisting of: the 1^{st} primer pair consisting of a base sequence represented by SEQ ID NO: 1 or 2; the 2^{nd} primer pair consisting of a base sequence represented by SEQ ID NO: 3 or 4; the 3^{rd} primer pair consisting of a base sequence represented by SEQ ID NO: 5 or 6; the 4^{th} primer pair consisting of a base sequence represented by SEQ ID NO: 7 or 8; the 5^{th} primer pair consisting of a base sequence represented by SEQ ID NO: 9 or 10; the 6^{th} primer pair consisting of a base sequence represented by SEQ ID NO: 11 or 12; the 7^{th} primer pair consisting of a base sequence represented by SEQ ID NO: 13 or 14; the 8^{th} primer pair consisting of a base sequence represented by SEQ ID NO: 15 or 16; the 9^{th} primer pair consisting of a base sequence represented by SEQ ID NO: 17 or 18; the 10^{th} primer pair consisting of a base sequence represented by SEQ ID NO: 19 or 20; the 11^{th} primer pair consisting of a base sequence represented by SEQ ID NO: 21 or 22; the 12^{th} primer pair consisting of a base sequence represented by SEQ ID NO: 23 or 24; the 13^{th} primer pair consisting of a base sequence represented by SEQ ID NO: 25 or 26; the 14^{th} primer pair consisting of a base sequence represented by SEQ ID NO: 27 or 28; the 15^{th} primer pair consisting of a base sequence represented by SEQ ID NO: 29 or 30; the 16^{th} primer pair consisting of a base sequence represented by SEQ ID NO: 31 or 32; the 17^{th} primer pair consisting of a base sequence represented by SEQ ID NO: 33 or 34; the 18^{th} primer pair consisting of a base sequence represented by SEQ ID NO: 35 or 36; the 19^{th} primer pair consisting of a base sequence represented by SEQ ID NO: 37 or 38; the 20^{th} primer pair consisting of a base sequence represented by SEQ ID NO: 39 or 40; the 21^{st} primer pair consisting of a base sequence represented by SEQ ID NO: 41 or 42; the 22^{nd} primer pair consisting of a base sequence represented by SEQ ID NO: 43 or 44; the 23^{rd} primer pair consisting of a base sequence represented by SEQ ID NO: 45 or 46; the 24^{th} primer pair consisting of a base sequence represented by SEQ ID NO: 47 or 48; the 25^{th} primer pair consisting of a base sequence represented by SEQ ID NO: 49 or 50; the 26^{th} primer pair consisting of a base sequence represented by SEQ ID NO: 51 or 52; the 27^{th} primer pair consisting of a base sequence represented by SEQ ID NO: 53 or 54; the 28^{th} primer pair consisting of a base sequence represented by SEQ ID NO: 55 or 56; the 29^{th} primer pair consisting of a base sequence represented by SEQ ID NO: 57 or 58; the 30^{th} primer pair consisting of a base sequence represented by SEQ ID NO: 59 or 60; the 31^{st} primer pair consisting of a base sequence represented by SEQ ID NO: 61 or 62; the 32^{nd} primer pair consisting of a base sequence represented by SEQ ID NO: 63 or 64; the 33^{rd} primer pair consisting of a base sequence represented by SEQ ID NO: 65 or 66; the 34^{th} primer pair consisting of a base sequence represented by SEQ ID NO: 67 or 68; the 35^{th} primer pair consisting of a base sequence represented by SEQ ID NO: 69 or 70; the 36^{th} primer pair consisting of a base sequence represented by SEQ ID NO: 71 or 72; the 37^{th} primer pair consisting of a base sequence represented by SEQ ID NO: 73 or 74; the 38^{th} primer pair consisting of a base sequence represented by SEQ ID NO: 75 or 76; and the 39^{th} primer pair consisting of a base sequence represented by SEQ ID NO: 77 or 78.

In addition, the present invention provides a composition for detecting food poisoning bacteria, the composition comprising the primer set.

In addition, the present invention provides a next-generation sequencing panel for detecting food poisoning bacteria, the panel comprising the primer set.

In addition, the present invention provides a kit for detecting food poisoning bacteria, the kit comprising the primer set.

In addition, the present invention provides a next-generation sequencing method, comprising the steps of: amplifying a target sequence by using the primer set and a sample; preparing a library by using the amplified sequence; and performing a next-generation sequencing analysis by using the prepared library.

In addition, the present invention provides a method for detecting food poisoning bacteria, the method comprising the step of performing an amplification reaction by using the primer set and a sample.

With the primer sets according to the present invention, 16 kinds of food poisoning bacteria can be detected at one time, for a shorter time, and with high accuracy, and the primer sets can be useful for simultaneous detection of food poisoning bacteria.

### BRIEF DESCRIPTION OF DRAWINGS

FIG. 1 is an agarose gel electrophoresis analysis photograph that shows showing the result of detecting Bacillus cereus by using the 1^{st} primer pair according to the present invention (M: DNA marker, 1: Campylobacter jejuni, 2: Campylobacter coli, 3: Clostridium perfringens, 4: Vibrio parahaemolyticus, 5: Vibrio vulnificus, 6: Vibrio cholerae, 7: Salmonella typhimurium, 8: Staphylococcus aureus, 9: Bacillus cereus, 10: Yersinia enterocolitica, 11: Listeria monocytogenes, 12: EHEC, 13: ETEC, 14: EPEC, 15: EIEC, 16: EAEC, and N: no template).

FIG. 2 is an agarose gel electrophoresis analysis photograph that shows the result of detecting Bacillus cereus by using the 2^{nd} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: EAEC, 9: ETEC, 10: EPEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 3 is an agarose gel electrophoresis photograph that shows the result of detecting Campylobacter by using the 3^{rd} primer pair according to the present invention (M: DNA marker, 1: Campylobacter jejuni, 2: Campylobacter coli, 3: Clostridium perfringens, 4: Vibrio parahaemolyticus, 5: Vibrio vulnificus, 6: Vibrio cholerae, 7: Salmonella typhimurium, 8: Staphylococcus aureus, 9: Bacillus cereus, 10: Yersinia enterocolitica, 11: Listeria monocytogenes, 12: EHEC, 13: ETEC, 14: EPEC, 15: EIEC, 16: EAEC, and N: no template).

FIG. 4 is an agarose gel electrophoresis photograph that shows the result of detecting Campylobacter coli by using the 4^{th} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: EAEC, 9: ETEC, 10: EPEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 5 is an agarose gel electrophoresis analysis photograph that shows the result of detecting Campylobacter coli by using the 5^{th} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: EAEC, 9: ETEC, 10: EPEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 6 is an agarose gel electrophoresis photograph that shows the result of detecting Campylobacter jejuni by using the 6^{th} primer pair according to the present invention (M: DNA marker, 1: Campylobacter jejuni, 2: Campylobacter coli, 3: Clostridium perfringens, 4: Vibrio parahaemolyticus, 5: Vibrio vulnificus, 6: Vibrio cholerae, 7: Salmonella typhimurium, 8: Staphylococcus aureus, 9: Bacillus cereus, 10: Yersinia enterocolitica, 11: Listeria monocytogenes, 12: EHEC, 13: ETEC, 14: EPEC, 15: EIEC, 16: EAEC, and N: no template).

FIG. 7 is an agarose gel electrophoresis photograph that shows the result of detecting Campylobacter jejuni by using the 7^{th} primer pair according to the present invention (M: DNA marker, 1: Campylobacter jejuni, 2: Campylobacter coli, 3: Clostridium perfringens, 4: Vibrio cholerae, 5: Vibrio parahaemolyticus, 6: Vibrio vulnificus, 7: Salmonella typhimurium, 8: Listeria monocytogenes, 9: Bacillus cereus, 10: Yersinia enterocolitica, 11: Staphylococcus aureus, 12: EHEC, 13: ETEC, 14: EPEC, 15: EIEC, 16: EAEC, and N: no template).

FIG. 8 is an agarose gel electrophoresis photograph that shows the result of detecting Clostridium perfringens by using the 8^{th} primer pair according to the present invention (M: DNA marker, 1: Campylobacter jejuni, 2: Campylobacter coli, 3: Clostridium perfringens, 4: Vibrio cholerae, 5: Vibrio parahaemolyticus, 6: Vibrio vulnificus, 7: Salmonella typhimurium, 8: Listeria monocytogenes, 9: Bacillus cereus, 10: Yersinia enterocolitica, 11: Staphylococcus aureus, 12: EHEC, 13: ETEC, 14: EPEC, 15: EIEC, 16: EAEC, and N: no template).

FIG. 9 is an agarose gel electrophoresis analysis photograph that shows the result of detecting Clostridium perfringens using the 9^{th} primer pair according to the present invention (M: DNA marker, 1: Campylobacter jejuni, 2: Campylobacter coli, 3: Clostridium perfringens, 4: Vibrio parahaemolyticus, 5: Vibrio vulnificus, 6: Vibrio cholerae, 7: Salmonella typhimurium, 8: Staphylococcus aureus, 9: Bacillus cereus, 10: Yersinia enterocolitica, 11: Listeria monocytogenes, 12: EHEC, 13: ETEC, 14: EPEC, 15: EIEC, 16: EAEC, and N: no template).

FIG. 10 is an agarose gel electrophoresis photograph that shows the result of detecting Clostridium perfringens by using the 10^{th} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: EAEC, 9: ETEC, 10: EPEC, 11: Salmonella typhimurium, 12: Vibrio vulnificus, 13: Vibrio cholerae, 14: Vibrio parahaemolyticus, 15: Campylobacter jejuni, 16: Campylobacter coli).

FIG. 11 is an agarose gel electrophoresis analysis photograph that shows the result of detecting EAEC by using the 11^{th} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: EAEC, 9: ETEC, 10: EPEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 12 is an agarose gel electrophoresis analysis photograph that shows the result of detecting EHEC by using the 12^{th} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: EAEC, 9: ETEC, 10: EPEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 13 is an Agarose gel electrophoresis analysis photograph that shows the result of detecting EHEC by using the 13^{th} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: EAEC, 9: ETEC, 10: EPEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 14 is an agarose gel electrophoresis analysis photograph that shows the result of detecting EHEC by using the 14^{th} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: EAEC, 9: ETEC, 10: EPEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 15 is an agarose gel electrophoresis analysis photograph that shows the result of detecting EPEC by using the 15^{th} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: EAEC, 9: ETEC, 10: EPEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 16 is an agarose gel electrophoresis analysis photograph that shows the result of detecting EIEC by using the 16^{th} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: EAEC, 9: ETEC, 10: EPEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 17 is an agarose gel electrophoresis analysis photograph that shows the result of detecting EIEC by using the 17^{th} primer pair according to the present invention (M: DNA marker, 1: Campylobacter jejuni, 2: Campylobacter coli, 3: Clostridium perfringens, 4: Vibrio cholerae, 5: Vibrio parahaemolyticus, 6: Vibrio vulnificus, 7: Salmonella typhimurium, 8: Listeria monocytogenes, 9: Bacillus cereus, 10: Yeshonia enterocolitica, 11: Staphylococcus aureus, 12: EHEC, 13: ETEC, 14: EPEC, 15: EIEC, 16: EAEC, and N: no template).

FIG. 18 is an agarose gel electrophoresis analysis photograph that shows the result of detecting ETEC by using the 18^{th} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: EAEC, 9: ETEC, 10: EPEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 19 is an agarose gel electrophoresis photograph that shows the result of detecting Listeria monocytogenes by using the 19^{th} primer pair according to the present invention (M: DNA marker, 1: Campylobacter jejuni, 2: Campylobacter coli, 3: Clostridium perfringens, 4: Vibrio cholerae, 5: Vibrio parahaemolyticus, 6: Vibrio vulnificus, 7: Salmonella typhimurium, 8: Listeria monocytogenes, 9: Bacillus cereus, 10: Yersinia enterocolitica, 11: Staphylococcus aureus, 12: EHEC, 13: ETEC, 14: EPEC, 15: EIEC, 16: EAEC, and N: no template).

FIG. 20 is an agarose gel electrophoresis photograph that shows the result of detecting Listeria monocytogenes by using the 20^{th} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: EAEC, 9: ETEC, 10: EPEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 21 is an agarose gel electrophoresis photograph that shows the result of detecting Listeria monocytogenes by using the 21^{st} primer pair according to the present invention (M: DNA marker, 1: Campylobacter jejuni, 2: Campylobacter coli, 3: Clostridium perfringens, 4: Vibrio parahaemolyticus, 5: Vibrio vulnificus, 6: Vibrio cholerae, 7: Salmonella typhimurium, 8: Staphylococcus aureus, 9: Bacillus cereus, 10: Yersinia enterocolitica, 11: Listeria monocytogenes, 7: 12: EHEC, 13: ETEC, 14: EPEC, 15: EIEC, 16: EAEC, and N: no template).

FIG. 22 is an agarose gel electrophoresis analysis photograph that shows the result of detecting Salmonella typhimurium by using the 22^{nd} primer pair according to the present invention (M: DNA marker, 1: Campylobacter jejuni, 2: Campylobacter coli, 3: Clostridium perfringens, 4: Vibrio parahaemolyticus, 5: Vibrio vulnificus, 6: Vibrio cholerae, 7: Salmonella typhimurium, 8: Staphylococcus aureus, 9: Bacillus cereus, 10: Yersinia enterocolitica, 11: Listeria monocytogenes, 12: EHEC, 13: ETEC, 14: EPEC, 15: EIEC, 16: EAEC, and N: no template).

FIG. 23 is an agarose gel electrophoresis analysis photograph that shows the result of detecting Salmonella typhimurium by using the 23^{rd} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: EAEC, 9: ETEC, 10: EPEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 24 is an agarose gel electrophoresis analysis photograph that shows the result of detecting Staphylococcus aureus by using the 24^{th} primer pair according to the present invention (M: DNA marker, 1: Staphylococcus aureus, 2: Yersinia enterocolitica, 3: Bacillus cereus, 4: Listeria monocytogenes, 5: Clostridium perfringens, 6: EHEC, 7: EIEC, 8: Salmonella typhimurium, 9: Vibrio vulnificus, 10: Vibrio cholerae, 11: Vibrio parahaemolyticus, 12: Campylobacter jejuni, and 13: Campylobacter coli).

FIG. 25 is an agarose gel electrophoresis photograph that shows the result of detecting Staphylococcus aureus by using the 25^{th} primer pair according to the present invention (M: DNA marker, 1: Campylobacter jejuni, 2: Campylobacter coli, 3: Clostridium perfringens, 4: Vibrio cholerae, 5: Vibrio parahaemolyticus, 6: Vibrio vulnificus, 7: Salmonella typhimurium, 8: Staphylococcus aureus, 9: Listeria monocytogenes, 10: Bacillus cereus, 11: Yersinia enterocolitica, 12: EHEC, 13: ETEC, 14: EPEC, 15: EIEC, 16: EAEC, and N: no template).

FIG. 26 is an agarose gel electrophoresis photograph that shows the result of detecting Staphylococcus aureus by using the 26^{th} primer pair according to the present invention (M: DNA marker, 1: Campylobacter jejuni, 2: Campylobacter coli, 3: Clostridium perfringens, 4: Vibrio cholerae, 5: Vibrio parahaemolyticus, 6: Vibrio vulnificus, 7: Salmonella typhimurium, 8: Staphylococcus aureus, 9: Listeria monocytogenes, 10: Bacillus cereus, 11: Yesinia enterocolitica, 12: EHEC, 13: ETEC, 14: EPEC, 15: EIEC, 16: EAEC, and N: no template).

FIG. 27 is an agarose gel electrophoresis photograph that shows the result of detecting Staphylococcus aureus by using the 27^{th} primer pair according to the present invention (M: DNA marker, Campylobacter jejuni, 2: Campylobacter coli, 3: Clostridium perfringens, 4: Vibrio cholerae, 5: Vibrio parahaemolyticus, 6: Vibrio vulnificus, 7: Salmonella typhimurium, 8: Staphylococcus aureus, 9: Listeria monocytogenes, 10: Bacillus cereus, 11: Yersinia enterocolitica, 12: EHEC, 13: ETEC, 14: EPEC, 15: EIEC, 16: EAEC, and N: no template).

FIG. 28 is an agarose gel electrophoresis analysis photograph that shows the result of detecting Staphylococcus aureus by using the 28^{th} primer pair according to the present invention (M: DNA marker, 1: Campylobacter jejuni, 2: Campylobacter coli, 3: Clostridium perfringens, 4: Vibrio cholerae, 5: Vibrio parahaemolyticus, 6: Vibrio vulnificus, 7: Salmonella typhimurium, 8: Staphylococcus aureus, 9: Listeria monocytogenes, 10: Bacillus cereus, 11: Yersinia enterocolitica, 12: EHEC, 13: ETEC, 14: EPEC, 15: EIEC, 16: EAEC, and N: no template).

FIG. 29 is an agarose gel electrophoresis photograph that shows the result of detecting Vibrio cholerae by using the 29^{th} primer pair according to the present invention (M: DNA marker, 1: Staphylococcus aureus (ATCC25923), 2: Staphylococcus aureus (ATCC23235), 3: Staphylococcus aureus (CCARM), 4: Staphylococcus aureus (NewMan), 5: Yersinia enterocolitica, 6: Bacillus cereus, 7: Listeria monocytogenes, 8: Clostridium perfringens, 9: EHEC, 10: EIEC, 11: EAEC, 12: ETEC, 13: EPEC, 14: Salmonella typhimurium, 15: Vibrio vulnificus, 16: Vibrio cholerae, 17: Vibrio parahaemolyticus, 18: Campylobacter jejuni, and 19: Campylobacter coli).

FIG. 30 is an agarose gel electrophoresis photograph that shows the result of detecting Vibrio cholerae by using the 30^{th} primer pair according to the present invention (M: DNA marker, 1: Staphylococcus aureus (ATCC25923), 2: Staphylococcus aureus (ATCC23235), 3: Staphylococcus aureus (CCARM), 4: Staphylococcus aureus (NewMan), 5: Yersinia enterocolitica, 6: Bacillus cereus, 7: Listeria monocytogenes, 8: Clostridium perfringens, 9: EHEC, 10: EIEC, 11: EAEC, 12: ETEC, 13: EPEC, 14: Salmonella typhimurium, 15: Vibrio vulnificus, 16: Vibrio cholerae, 17: Vibrio parahaemolyticus, 18: Campylobacter jejuni, and 19: Campylobacter coli).

FIG. 31 is an agarose gel electrophoresis photograph that shows the result of detecting Vibrio cholerae by using the 31^{st} primer pair according to the present invention (M: DNA marker, 1: Campylobacter jejuni, 2: Campylobacter coli, 3: Clostridium perfringens, 4: Vibrio parahaemolyticus, 5: Vibrio vulnificus, 6: Vibrio cholerae, 7: Salmonella typhimurium, 8: Staphylococcus aureus, 9: Bacillus cereus, 10: Yersinia enterocolitica, 11: Listeria monocytogenes, 12: EHEC, 13: ETEC, 14: EPEC, 15: EIEC, 16: EAEC, and N: no template).

FIG. 32 is an agarose gel electrophoresis photograph that shows the result of detecting Vibrio parahaemolyticus by using the 32^{nd} primer pair according to the present invention (M: DNA marker, 1: Staphylococcus aureus (CCARM), 2: Staphylococcus aureus (NewMan), 3: Staphylococcus aureus (ATCC25923), 4: Staphylococcus aureus (ATCC23235), 5: Yersinia enterocolitica, 6: Bacillus cereus, 7: Listeria monocytogenes, 8: Clostridium perfringens, 9: EHEC, 10: EIEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 33 is an agarose gel electrophoresis photograph that shows the result of detecting Vibrio parahaemolyticus by using the 33^{rd} primer pair according to the present invention (M: DNA marker, 1: Campylobacter jejuni, 2: Campylobacter coli, 3: Clostridium perfringens, 4: Vibrio cholerae, 5: Vibrio parahaemolyticus, 6: Vibrio vulnificus, 7: Salmonella typhimurium, 8: Listeria monocytogenes, 9: Bacillus cereus, 10: Yersinia enterocolitica, 11: Staphylococcus aureus, 12: EHEC, 13: ETEC, 14: EPEC, 15: EIEC, 16: EAEC, and N: no template).

FIG. 34 is an agarose gel electrophoresis photograph that shows the result of detecting Vibrio parahaemolyticus by using the 34^{th} primer pair according to the present invention (M: DNA marker, 1: Campylobacter jejuni, 2: Campylobacter coli, 3: Clostridium perfringens, 4: Vibrio parahaemolyticus, 5: Vibrio vulnificus, 6: Vibrio cholerae, 7: Salmonella typhimurium, 8: Staphylococcus aureus, 9: Bacillus cereus, 10: Yeshnia enterocolitica, 11: Listeria monocytogenes, 12: EHEC, 13: ETEC, 14: EPEC, 15: EIEC, 16: EAEC, and N: no template).

FIG. 35 is an agarose gel electrophoresis photograph that shows the result of detecting Vibrio parahaemolyticus by using the 35^{th} primer pair according to the present invention (M: DNA marker, 1: Campylobacter jejuni, 2: Campylobacter coli, 3: Clostridium perfringens, 4: Vibrio parahaemolyticus, 5: Vibrio vulnificus, 6: Vibrio cholerae, 7: Salmonella typhimurium, 8: Staphylococcus aureus, 9: Bacillus cereus, 10: Yersinia enterocolitica, 11: Listeria monocytogenes, 12: EHEC, 13: ETEC, 14: EPEC, 15: EIEC, 16: EAEC, and N: no template).

FIG. 36 is an agarose gel electrophoresis photograph that shows the result of detecting Vibrio vulnificus by using the 36^{th} primer pair according to the present invention (M: DNA marker, 1: Staphylococcus aureus (CCARM), 2: Staphylococcus aureus (NewMan), 3: Staphylococcus aureus (ATCC25923), 4: Staphylococcus aureus (ATCC23235), 5: Yersinia enterocolitica, 6: Bacillus cereus, 7: Listeria monocytogenes, 8: Clostridium perfringens, 9: EHEC, 10: EIEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 37 is an agarose gel electrophoresis photograph that shows the result of detecting Vibrio vulnificus by using the 37^{th} primer pair according to the present invention (M: DNA marker, 1: Staphylococcus aureus (CCARM), 2: Staphylococcus aureus (NewMan), 3: Staphylococcus aureus (ATCC25923), 4: Staphylococcus aureus (ATCC23235), 5: Yersinia enterocolitica, 6: Bacillus cereus, 7: Listeria monocytogenes, 8: Clostridium perfringens, 9: EHEC, 10: EIEC, 11: Salmonella typhimurium, 12: Vibrio vulnificus, 13: Vibrio cholerae, 14: Vibrio parahaemolyticus, 15: Campylobacter jejuni, and 16: Campylobacter coli).

FIG. 38 is an agarose gel electrophoresis analysis photograph that shows the result of detecting Yersinia enterocolitica by using the 38^{th} primer pair according to the present invention (M: DNA marker, 1: Campylobacter jejuni, 2: Campylobacter coli, 3: Clostridium perfringens, 4: Vibrio cholerae, 5: Vibrio parahaemolyticus, 6: Vibrio vulnificus, 7: Salmonella typhimurium, 8: Listeria monocytogenes, 9: Bacillus cereus, 10: Yersinia enterocolitica, 11: Staphylococcus aureus, 12: EHEC, 13: EAEC, 14: EPEC, 15: EIEC, 16: ETEC, and N: no template).

FIG. 39 is an agarose gel electrophoresis analysis photograph that shows the result of detecting Yersinia enterocolitica by using the 39^{th} primer pair according to the present invention (M: DNA marker, 1: Campylobacter jejuni, 2: Campylobacter coli, 3: Clostridium perfringens, 4: Vibrio cholerae, 5: Vibrio parahaemolyticus, 6: Vibrio vulnificus, 7: Salmonella typhimurium, 8: Listeria monocytogenes, 9: Bacillus cereus, 10: Staphylococcus aureus, 11: Yersinia enterocolitica, 12: EHEC, 13: ETEC, 14: EPEC, 15: EIEC, 16: EAEC, and N: no template).

FIG. 40 is a result of performing a PCR, in accordance with an embodiment of the present invention, by mixing all primer pairs according to the present invention and food poisoning bacteria genes to perform NGS.

FIG. 41 is a view showing a portion of the resulting data obtained by performing NGS according to an embodiment of the present invention.

FIG. 42 is a view showing the number of target sites detected by performing NGS according to an embodiment of the present invention.

FIG. 43 is a schematic view showing a food poisoning bacteria detection method according to an embodiment of the present invention.

### DETAILED DESCRIPTION

Hereinafter, the present invention will be described in detail.

The present invention provides a primer set for detecting food poisoning bacteria, the primer set comprising at least one selected from the group consisting of: the 1^{st} primer pair consisting of a base sequence represented by SEQ ID NO: 1 or 2; the 2^{nd} primer pair consisting of a base sequence represented by SEQ ID NO: 3 or 4; the 3^{rd} primer pair consisting of a base sequence represented by SEQ ID NO: 5 or 6; the 4^{th} primer pair consisting of a base sequence represented by SEQ ID NO: 7 or 8; the 5^{th} primer pair consisting of a base sequence represented by SEQ ID NO: 9 or 10; the 6^{th} primer pair consisting of a base sequence represented by SEQ ID NO: 11 or 12; the 7^{th} primer pair consisting of a base sequence represented by SEQ ID NO: 13 or 14; the 8^{th} primer pair consisting of a base sequence represented by SEQ ID NO: 15 or 16; the 9^{th} primer pair consisting of a base sequence represented by SEQ ID NO: 17 or 18; the 10^{th} primer pair consisting of a base sequence represented by SEQ ID NO: 19 or 20; the 11^{th} primer pair consisting of a base sequence represented by SEQ ID NO: 21 or 22; the 12^{th} primer pair consisting of a base sequence represented by SEQ ID NO: 23 or 24; the 13^{th} primer pair consisting of a base sequence represented by SEQ ID NO: 25 or 26; the 14^{th} primer pair consisting of a base sequence represented by SEQ ID NO: 27 or 28; the 15^{th} primer pair consisting of a base sequence represented by SEQ ID NO: 29 or 30; the 16^{th} primer pair consisting of a base sequence represented by SEQ ID NO: 31 or 32; the 17^{th} primer pair consisting of a base sequence represented by SEQ ID NO: 33 or 34; the 18^{th} primer pair consisting of a base sequence represented by SEQ ID NO: 35 or 36; the 19^{th} primer pair consisting of a base sequence represented by SEQ ID NO: 37 or 38; the 20^{th} primer pair consisting of a base sequence represented by SEQ ID NO: 39 or 40; the 21^{st} primer pair consisting of a base sequence represented by SEQ ID NO: 41 or 42; the 22^{nd} primer pair consisting of a base sequence represented by SEQ ID NO: 43 or 44; the 23^{rd} primer pair consisting of a base sequence represented by SEQ ID NO: 45 or 46; the 24^{th} primer pair consisting of a base sequence represented by SEQ ID NO: 47 or 48; the 25^{th} primer pair consisting of a base sequence represented by SEQ ID NO: 49 or 50; the 26^{th} primer pair consisting of a base sequence represented by SEQ ID NO: 51 or 52; the 27^{th} primer pair consisting of a base sequence represented by SEQ ID NO: 53 or 54; the 28^{th} primer pair consisting of a base sequence represented by SEQ ID NO: 55 or 56; the 29^{th} primer pair consisting of a base sequence represented by SEQ ID NO: 57 or 58; the 30^{th} primer pair consisting of a base sequence represented by SEQ ID NO: 59 or 60; the 31^{st} primer pair consisting of a base sequence represented by SEQ ID NO: 61 or 62; the 32^{nd} primer pair consisting of a base sequence represented by SEQ ID NO: 63 or 64; the 33^{rd} primer pair consisting of a base sequence represented by SEQ ID NO: 65 or 66; the 34^{th} primer pair consisting of a base sequence represented by SEQ ID NO: 67 or 68; the 35^{th} primer pair consisting of a base sequence represented by SEQ ID NO: 69 or 70; the 36^{th} primer pair consisting of a base sequence represented by SEQ ID NO: 71 or 72; the 37^{th} primer pair consisting of a base sequence represented by SEQ ID NO: 73 or 74; the 38^{th} primer pair consisting of a base sequence represented by SEQ ID NO: 75 or 76; and the 39^{th} primer pair consisting of a base sequence represented by SEQ ID NO: 77 or 78.

In one aspect of the present invention, the primer set may comprise:
a) at least one primer pair selected from the group consisting of the 1^{st} primer pair consisting of a base sequence represented by SEQ ID NO: 1 or 2 and the 2^{nd} primer pair consisting of a base sequence represented by SEQ ID NO: 3 or 4;
b) the 3^{rd} primer pair consisting of a base sequence represented by SEQ ID NO: 5 or 6;
c) at least one primer pair selected from the group consisting of the 4^{th} primer pair consisting of a base sequence represented by SEQ ID NO: 7 or 8 and the 5^{th} primer pair consisting of a base sequence represented by SEQ ID NO: 9 or 10;
d) at least one primer pair selected from the group consisting of the 6^{th} primer pair consisting of a base sequence represented by SEQ ID NO: 11 or 12 and the 7^{th} primer pair consisting of a base sequence represented by SEQ ID NO: 13 or 14;
e) at least one primer pair selected from the group consisting of the 8^{th} primer pair consisting of a base sequence represented by SEQ ID NO: 15 or 16, the 9^{th} primer pair consisting of a base sequence represented by SEQ ID NO: 17 or 18, and the 10^{th} primer pair consisting of a base sequence represented by SEQ ID NO: 19 or 20;
f) the 11^{th} primer pair consisting of a base sequence represented by SEQ ID NO: 21 or 22;
g) at least one primer pair selected from the group consisting of the 12^{th} primer pair consisting of a base sequence represented by SEQ ID NO: 23 or 24, the 13^{th} primer pair consisting of a base sequence represented by SEQ ID NO: 25 or 26, and the 14^{th} primer pair consisting of a base sequence represented by SEQ ID NO: 27 or 28;
h) the 15^{th} primer pair consisting of a base sequence represented by SEQ ID NO: 29 or 30;
i) at least one primer pair selected from the group consisting of the 16^{th} primer pair consisting of a base sequence represented by SEQ ID NO: 31 or 32 and the 17^{th} primer pair consisting of a base sequence represented by SEQ ID NO: 33 or 34;
j) the 18^{th} primer pair consisting of a base sequence represented by SEQ ID NO: 35 or 36;
k) at least one primer pair selected from the group consisting of the 19^{th} primer pair consisting of a base sequence represented by SEQ ID NO: 37 or 38, the 20^{th} primer pair consisting of a base sequence represented by SEQ ID NO: 39 or 40, and the 21^{st} primer pair consisting of a base sequence represented by SEQ ID NO: 41 or 42;
l) at least one primer pair selected from the group consisting of the 22^{nd} primer pair consisting of a base sequence represented by SEQ ID NO: 43 or 44 and the 23^{rd} primer pair consisting of a base sequence represented by SEQ ID NO: 45 or 46;
m) at least one primer pair selected from the group consisting of the 24^{th} primer pair consisting of a base sequence represented by SEQ ID NO: 47 or 48, the 25^{th} primer pair consisting of a base sequence represented by SEQ ID NO: 49 or 50, the 26^{th} primer pair consisting of a base sequence represented by SEQ ID NO: 51 or 52, the 27^{th} primer pair consisting of a base sequence represented by SEQ ID NO: 53 or 54, and the 28^{th} primer pair consisting of a base sequence represented by SEQ ID NO: 55 or 56;
n) at least one primer pair selected from the group consisting of the 29^{th} primer pair consisting of a base sequence represented by SEQ ID NO: 57 or 58, the 30^{th} primer pair consisting of a base sequence represented by SEQ ID NO: 59 or 60, and the 31^{st} primer pair consisting of a base sequence represented by SEQ ID NO: 61 or 62;
o) at least one primer pair selected from the group consisting of the 32^{nd} primer pair consisting of a base sequence represented by SEQ ID NO: 63 or 64, the 33^{rd} primer pair consisting of a base sequence represented by SEQ ID NO: 65 or 66, the 34^{th} primer pair consisting of a base sequence represented by SEQ ID NO: 67 or 68, and the 35^{th} primer pair consisting of a base sequence represented by SEQ ID NO: 69 or 70;
p) at least one primer pair selected from the group consisting of the 36^{th} primer pair consisting of a base sequence represented by SEQ ID NO: 71 or 72 and the 37^{th} primer pair consisting of a base sequence represented by SEQ ID NO: 73 or 74; and
q) at least one primer pair selected from the group consisting of the 38^{th} primer pair consisting of a base sequence represented by SEQ ID NO: 75 or 76 and the 39^{th} primer pair consisting of a base sequence represented by SEQ ID NO: 77 or 78.

In another aspect of the present invention, the primer set may comprise:

the 1^{st} primer pair consisting of a base sequence represented by SEQ ID NO: 1 or 2;

the 2^{nd} primer pair consisting of a base sequence represented by SEQ ID NO: 3 or 4;

the 3^{rd} primer pair consisting of a base sequence represented by SEQ ID NO: 5 or 6;

the 4^{th} primer pair consisting of a base sequence represented by SEQ ID NO: 7 or 8;

the 5^{th} primer pair consisting of a base sequence represented by SEQ ID NO: 9 or 10;

the 6^{th} primer pair consisting of a base sequence represented by SEQ ID NO: 11 or 12;

the 7^{th} primer pair consisting of a base sequence represented by SEQ ID NO: 13 or 14;

the 8^{th} primer pair consisting of a base sequence represented by SEQ ID NO: 15 or 16;

the 9^{th} primer pair consisting of a base sequence represented by SEQ ID NO: 17 or 18;

the 10^{th} primer pair consisting of a base sequence represented by SEQ ID NO: 19 or 20;

the 11^{th} primer pair consisting of a base sequence represented by SEQ ID NO: 21 or 22;

the 12^{th} primer pair consisting of a base sequence represented by SEQ ID NO: 23 or 24;

the 13^{th} primer pair consisting of a base sequence represented by SEQ ID NO: 25 or 26;

the 14^{th} primer pair consisting of a base sequence represented by SEQ ID NO: 27 or 28;

the 15^{th} primer pair consisting of a base sequence represented by SEQ ID NO: 29 or 30;

the 16^{th} primer pair consisting of a base sequence represented by SEQ ID NO: 31 or 32;

the 17^{th} primer pair consisting of a base sequence represented by SEQ ID NO: 33 or 34;

the 18^{th} primer pair consisting of a base sequence represented by SEQ ID NO: 35 or 36;

the 19^{th} primer pair consisting of a base sequence represented by SEQ ID NO: 37 or 38;

the 20^{th} primer pair consisting of a base sequence represented by SEQ ID NO: 39 or 40;

the 21^{st} primer pair consisting of a base sequence represented by SEQ ID NO: 41 or 42;

the 22^{nd} primer pair consisting of a base sequence represented by SEQ ID NO: 43 or 44;

the 23^{rd} primer pair consisting of a base sequence represented by SEQ ID NO: 45 or 46;

the 24^{th} primer pair consisting of a base sequence represented by SEQ ID NO: 47 or 48;

the 25^{th} primer pair consisting of a base sequence represented by SEQ ID NO: 49 or 50;

the 26^{th} primer pair consisting of a base sequence represented by SEQ ID NO: 51 or 52;

the 27^{th} primer pair consisting of a base sequence represented by SEQ ID NO: 53 or 54;

the 28^{th} primer pair consisting of a base sequence represented by SEQ ID NO: 55 or 56;

the 29^{th} primer pair consisting of a base sequence represented by SEQ ID NO: 57 or 58;

the 30^{th} primer pair consisting of a base sequence represented by SEQ ID NO: 59 or 60;

the 31^{st} primer pair consisting of a base sequence represented by SEQ ID NO: 61 or 62;

the 32^{nd} primer pair consisting of a base sequence represented by SEQ ID NO: 63 or 64;

the 33^{rd} primer pair consisting of a base sequence represented by SEQ ID NO: 65 or 66;

the 34^{th} primer pair consisting of a base sequence represented by SEQ ID NO: 67 or 68;

the 35^{th} primer pair consisting of a base sequence represented by SEQ ID NO: 69 or 70;

the 36^{th} primer pair consisting of a base sequence represented by SEQ ID NO: 71 or 72;

the 37^{th} primer pair consisting of a base sequence represented by SEQ ID NO: 73 or 74;

the 38^{th} primer pair consisting of a base sequence represented by SEQ ID NO: 75 or 76;

and

the 39^{th} primer pair consisting of a base sequence represented by SEQ ID NO: 77 or 78.

The base sequence of a primer constituting the primer set of the present invention, as long as the primer is specifically bound to a target location, may have sequence homology of 80% or more, 90% or more, 95% or more, 97% or more or 99% or more with respect to any of the sequences represented by SEQ ID NO: 1 to 78, and the primer may be a variant of any of the sequences represented by SEQ ID NO: 1 to 78. The variant may be formed by substituting, deleting, inserting, or removing one or more bases.

The term ‘food poisoning bacteria’ used herein refers to bacteria that may cause, via food or water, intoxication symptoms such as acute gastroenteritis and neurological disorders. The food poisoning bacteria may include both bacteria (toxin-producing bacteria) that can grow in food and produce a toxin causing food poisoning and bacteria that can, when it is taken into a subject, cause food poisoning.

The food poisoning bacteria may include all kinds of food poisoning bacteria known in the ordinary art, and it may be, for example, at least one selected from the group consisting of Bacillus, Campylobacter, Clostridium, Escherichia, Listeria, Salmonella, Staphylococcus, Vibrio, and Yersinia. Specifically, the food poisoning bacteria may be at least one selected from the group consisting of Bacillus cereus, Campylobacter coli, Campylobacter jejuni, Clostridium perfringens, enteroaggregative Escherichia coli (EAEC), enterohemorrhagic Escherichia coli (EHEC), enteropathogenic Escherichia coli (EPEC), enteroinvasive Escherichia coli (EIEC), enterotoxigenic Escherichia coli (ETEC), Listeria monocytogenes, Salmonella typhimurium, Staphylococcus aureus, Vibrio cholerae, Vibrio parahaemolyticus, Vibrio vulnificus and Yersinia enterocolitica.

For example, the primer set may comprise:
a) at least one primer pair selected from the group consisting of the 1^{st} primer pair consisting of a base sequence represented by SEQ ID NO: 1 or 2 and the 2^{nd} primer pair consisting of a base sequence represented by SEQ ID NO: 3 or 4, the at least one primer pair being capable of detecting Bacillus cereus;
b) the 3^{rd} primer pair consisting of a base sequence represented by SEQ ID NO: 5 or 6, the 3^{rd} primer pair being capable of detecting Campylobacter;
c) at least one primer pair selected from the group consisting of the 4^{th} primer pair consisting of a base sequence represented by SEQ ID NO: 7 or 8 and the 5^{th} primer pair consisting of a base sequence represented by SEQ ID NO: 9 or 10, the at least one primer pair being capable of detecting Campylobacter coli;
d) at least one primer pair selected from the group consisting of the 6^{th} primer pair consisting of a base sequence represented by SEQ ID NO: 11 or 12 and the 7^{th} primer pair consisting of a base sequence represented by SEQ ID NO: 13 or 14, the at least one primer pair being capable of detecting Campylobacter jejuni;
e) at least one primer pair selected from the group consisting of the 8^{th} primer pair consisting of a base sequence represented by SEQ ID NO: 15 or 16, the 9^{th} primer pair consisting of a base sequence represented by SEQ ID NO: 17 or 18, and the 10^{th} primer pair consisting of a base sequence represented by SEQ ID NO: 19 or 20, the at least one primer pair being capable of detecting Clostridium perfringens;
f) the 11^{th} primer pair consisting of a base sequence represented by SEQ ID NO: 21 or 22, the 11^{th} primer pair being capable of detecting enteroaggregative Escherichia coli (EAEC);
g) at least one primer pair selected from the group consisting of the 12^{th} primer pair consisting of a base sequence represented by SEQ ID NO: 23 or 24, the 13^{th} primer pair consisting of a base sequence represented by SEQ ID NO: 25 or 26, and the 14^{th} primer pair consisting of a base sequence represented by SEQ ID NO: 27 or 28, the at least one primer pair being capable of detecting enterohemorrhagic Escherichia coli (EHEC);
h) the 15^{th} primer pair consisting of a base sequence represented by SEQ ID NO: 29 or 30, the 15^{th} primer pair being capable of detecting enteropathogenic Escherichia coli (EPEC);
i) at least one primer pair selected from the group consisting of the 16^{th} primer pair consisting of a base sequence represented by SEQ ID NO: 31 or 32 and the 17^{th} primer pair consisting of a base sequence represented by SEQ ID NO: 33 or 34, the at least one primer pair being capable of detecting enteroinvasive Escherichia coli (EIEC);
j) the 18^{th} primer pair consisting of a base sequence represented by SEQ ID NO: 35 or 36, the 18^{th} primer pair being capable of detecting enterotoxigenic Escherichia coli (ETEC);
k) at least one primer pair selected from the group consisting of the 19^{th} primer pair consisting of a base sequence represented by SEQ ID NO: 37 or 38, the 20^{th} primer pair consisting of a base sequence represented by SEQ ID NO: 39 or 40, and the 21^{st} primer pair consisting of a base sequence represented by SEQ ID NO: 41 or 42, the at least one primer pair being capable of detecting Listeria monocytogenes;
l) at least one primer pair selected from the group consisting of the 22^{nd} primer pair consisting of a base sequence represented by SEQ ID NO: 43 or 44 and the 23^{rd} primer pair consisting of a base sequence represented by SEQ ID NO: 45 or 46, the at least one primer pair being capable of detecting Salmonella typhimurium;
m) at least one primer pair selected from the group consisting of the 24^{th} primer pair consisting of a base sequence represented by SEQ ID NO: 47 or 48, the 25^{th} primer pair consisting of a base sequence represented by SEQ ID NO: 49 or 50, the 26^{th} primer pair consisting of a base sequence represented by SEQ ID NO: 51 or 52, the 27^{th} primer pair consisting of a base sequence represented by SEQ ID NO: 53 or 54, and the 28^{th} primer pair consisting of a base sequence represented by SEQ ID NO: 55 or 56, the at least one primer pair being capable of detecting Staphylococcus aureus;
n) at least one primer pair selected from the group consisting of the 29^{th} primer pair consisting of a base sequence represented by SEQ ID NO: 57 or 58, the 30^{th} primer pair consisting of a base sequence represented by SEQ ID NO: 59 or 60, and the 31^{st} primer pair consisting of a base sequence represented by SEQ ID NO: 61 or 62, the at least one primer pair being capable of detecting Vibrio cholerae;
o) at least one primer pair selected from the group consisting of the 32^{nd} primer pair consisting of a base sequence represented by SEQ ID NO: 63 or 64, the 33^{rd} primer pair consisting of a base sequence represented by SEQ ID NO: 65 or 66, the 34^{th} primer pair consisting of a base sequence represented by SEQ ID NO: 67 or 68, and the 35^{th} primer pair consisting of a base sequence represented by SEQ ID NO: 69 or 70, the at least one primer pair being capable of detecting Vibrio parahaemolyticus;
p) at least one primer pair selected from the group consisting of the 36^{th} primer pair consisting of a base sequence represented by SEQ ID NO: 71 or 72 and the 37^{th} primer pair consisting of a base sequence represented by SEQ ID NO: 73 or 74, the at least one primer pair being capable of detecting Vibrio vulnificus; and
q) at least one primer pair selected from the group consisting of the 38^{th} primer pair consisting of a base sequence represented by SEQ ID NO: 75 or 76 and the 39^{th} primer pair consisting of a base sequence represented by SEQ ID NO: 77 or 78, the at least one primer pair being capable of detecting Yersinia enterocolitica.

Detection of food poisoning bacteria by using the primer set can be carried out by using a molecular detection method. The term ‘molecular detection’ refers to a method of detecting, by numerical values or images, changes occurring in cells at various molecular levels. The molecular detection generally refers to analysis of nucleic acids such as DNA or RNA, but it may include analysis of proteins and intracellular metabolom. Specifically, for example, the primer set according to the present invention can be used to detect food poisoning bacteria through DNA analysis.

The detection can be performed by amplifying a target gene present in a food poisoning bacteria genome. At this time, the amplification of the gene can be carried out in any way known in the art. For example, the gene amplification may be carried out by a polymerase chain reaction, a real-time polymerase chain reaction (real time PCR), or multiple polymerase chain reactions (multiplex PCR), and the like. In another aspect of the present invention, the detection can be carried out by a next-generation sequencing method.

The primer constituting the primer set may be a conjugate labeled with a detector such as a coloring enzyme, a fluorescent agent, a radioisotope, or a colloid. Examples of the coloring enzyme may include peroxidase, alkaline phosphatase, or acidic phosphatase. Examples of the fluorescent agent may include fluorescein thiourea (FTH), 7-acetoxycoumarin-3-yl, fluorescein-5-yl, fluorescein-6-yl, 2′,7′-dichlorofluorescein-5-yl, 2′,7′-dichlorofluorescein-6-yl, dihydrotetramethyllosamine-4-yl, tetramethyllodamine-5-yl, tetramethyllodamine-6-yl, 4,4-difluoro-5,7-dimethyl-4-bora-3a, 4a-diaza-s-indacene-3-ethyl or 4,4-difluoro-5,7-diphenyl-4-bora-3a, 4a-diaza-s-indacene-3-ethyl, Cy3, Cy5, poly L-lysine-fluorescein iso thiocyanate (FITC), rhodamine-B-isothiocyanate (RITC), Phycoerythrin (PE), or rhodamine.

In addition, the present invention provides a composition for detecting food poisoning bacteria, the composition comprising the primer set.

The primers that may be included in the composition according to the present invention and the food poisoning bacteria that can be detected by using the primers may have the characteristics described above.

In addition, the composition may further comprise a ligand that can be specifically bound to the primer included in the primer set according to the present invention. The ligand may be a conjugate in which detectors such as coloring enzymes, fluorescent agents, radioisotopes, or colloids are labeled, and the ligand may be treated with streptavidin or avidin. The composition according to the present invention may further comprise distilled water or a buffer to ensure structural stability.

In addition, the present invention provides a next-generation sequencing panel for detecting food poisoning bacteria, the panel comprising the primer set.

The primers that may be included in the panel according to the present invention and the food poisoning bacteria that can be detected by using the primers may have the characteristics described above.

The term ‘next-generation sequencing (NGS)’ used herein refers to a method that can sequence bases of a gene at a high speed, in which a gene can be put into many pieces, the pieces can be read at the same time, and the thus-obtained data can be combined by using bioinformatic techniques, thereby making it possible to quickly decipher the genetic information. Examples of the next-generation sequencing may include single molecule real-time sequencing, ion semiconductor/ion torrent sequencing, pyrosequencing, sequencing by synthesis, sequencing by ligation, nanopore sequencing, and the like.

In addition, the present invention provides a kit for detecting food poisoning bacteria, the kit comprising the primer set.

The primers that may be included in the kit according to the present invention and the food poisoning bacteria that can be detected by using the primers may have the characteristics described above.

The kit may be coupled to a solid substrate to facilitate subsequent steps, such as washing the primer or dissembling the kit. Examples of the solid substrate may include synthetic resins, nitrocellulose, glass substrates, metal substrates, glass fibers, microspheres, and microbeads. In addition, examples of the synthetic resins may include polyesters, polyvinyl chlorides, polystyrenes, polypropylenes, PVDFs, and nylons.

In addition, the kit may be prepared by a conventional manufacturing method known in the art, and it may further comprise buffers, stabilizers, inert proteins, and the like. A high throughput screening (HTS) system in which a fluorescence level of a fluorescent agent used as a detector is measured or the radiation level of a radiation isotope used as a detector is measured, a surface plasmon resonance (SPR) method in which a surface plasmon resonance change is measured in real time without using a detector, or a surface plasmon resonance imaging (SPRI) method in which data from the SPR system is converted to image data can be used.

The type of food poisoning bacteria that can be detected by using the kit is as described above. On the other hand, in another aspect of the present invention, a next-generation sequencing method may be used to detect food poisoning bacteria by using the kit. In order to detect food poisoning bacteria by the next-generation sequencing method, additional reagents commonly used in the next-generation sequencing may further be comprised in the kit. For example, the kit may further comprise a reagent, such as a polymerase, dNTP, and the like, for amplifying a target site. In addition, the kit may further comprise an A-tail, an adapter, a bead, a buffer, and so on for purifying an amplified product and using the amplified product to prepare a library for us in next-generation sequencing. A person having ordinary skill can choose appropriate quantities of the additional reagents.

In addition, the present invention provides a next-generation sequencing method comprising the steps of amplifying a target sequence by using a sample and the primer set; preparing a library by using the amplified sequence; and performing a next-generation sequencing analysis by using the prepared library.

The primers that may be used in the next-generation sequencing method according to the present invention and the food poisoning bacteria that can be detected by using the primers may have the characteristics described above.

On the other hand, the next-generation sequencing analysis may also have the characteristics as described above, which can be performed by any method known in the art. The method may be suitably modified by a person having ordinary skill in the art.

Any sample can be used for the purpose of detecting food poisoning bacteria. For example, the sample may be any food material such as vegetables, meat, fish, seafood, etc. or any food prepared by using the food materials. On the other hand, the sample may be used to diagnose a subject with food poisoning. The subject may be, for example, mammals, including humans.

In addition, before being applied to the detection method according to the present invention, the sample can be suitably pretreated by using various methods such as anion exchange chromatography, affinity chromatography, size exclusion chromatography, liquid chromatography, continuous extraction, centrifugation, gel electrophoresis, and so on.

Furthermore, the present invention provides a method for detecting food poisoning bacteria, the method comprising the step of performing an amplification reaction by using the primer set and the sample.

The primers that may be used in the methods according to the present invention and the food poisoning bacteria that can be detected by using the primers may have the characteristics described above. The amplification may be performed in the same manner as described above. The amplification may be performed by using a reaction product prepared in a conventional manner. In one aspect of the present invention, the next-generation sequencing method can be used, which sequencing method can be carried out in a conventional manner. In addition, this method can be appropriately modified by a person having ordinary skill in the art.

The sample used in the detection methods according to the present invention may have the characteristics as described above, and the sample may be pretreated as described above.

### EXAMPLES

Hereinafter, the present invention will be described in detail by the following embodiments, but, the present invention is not limited to the embodiments as the embodiments are only intended to illustrate the present invention. Any subject matter that has the same structure as the subject matter claimed in the following claim section and have the same effect as the effect resulting from the subject matter claimed in the following claim section should be included in the technical scope of the present invention.

### Example 1. Preparation of Primer Set

A primer set for simultaneously detecting 16 types of food poisoning bacteria by using next-generation sequencing (NGS) was prepared.

First, from the gene sequences of 16 types of food poisoning bacteria, a region that has the highest homology within the same species of food poisoning bacteria and does not have homology between different species of food poisoning bacteria was selected, and a primer corresponding to the selected region was prepared. The primer comprised 20 to 23 bases, and the primer was prepared such that the PCR product obtained through PCR amplification was 150 to 250 bp long, had GC content of 40 to 60%, and had a Tm value of 53 to 60° C. In addition, the self-compatibility was at least 4. The thus-prepared primer set is shown in Table 1 below.

TABLE 1

| Strains | Primer | Sequence | SEQ ID NO. |
|---|---|---|---|
| Bacillus | 1-F | GCG CTC TTC TAA AGT CTC AC | SEQ ID NO: 1 |
| cereus | | | |
| | 1-R | CGA AAT TAG CCC AGT AGC AC | SEQ ID NO: 2 |
| | 2-F | (GAA CTG CTG GTA CAA CAC CTG | SEQ ID NO: 3 |
| | 2-R | TCT GCA CTA ATG AAC TGA CCG | SEQ ID NO: 4 |
| Campylobacter | 3-F | CCG CTT TTG GAC CTC AAT CTC GC | SEQ ID NO: 5 |
| spp. | | | |
| | 3-R | GCC TTG TGC GCG TTC TTT ATT GCC | SEQ ID NO: 6 |
| Campylobacter | 4-F | GCA AGA ATT ACC TTT GGT TGA | SEQ ID NO: 7 |
| coli | | | |
| | 4-R | CCT CCA TTT GCT TGT ATT CAT | SEQ ID NO: 8 |
| | 5-F | CCT ACG ACT TGT TTG ATT TGG | SEQ ID NO: 9 |
| | 5-R | CCA CAG AAT GAC CTC CAT TAG | SEQ ID NO: 10 |
| Campylobacter | 6-F | TGG AGT TGT TGT GGG GCT TC | SEQ ID NO: 11 |
| jejuni | | | |
| | 6-R | TGA TGC GTT CTT TTG CAC CC | SEQ ID NO: 12 |
| | 7-F | TTA TGG CGA GGG ACT TGG AGC | SEQ ID NO: 13 |
| | 7-R | AGA ATA ATG CGC CGT TGG ACG | SEQ ID NO: 14 |
| Clostridium | 8-F | ATG TTA CTG CCG TTG ATA GCG | SEQ ID NO: 15 |
| perfringens | | | |
| | 8-R | GCT GCA TAA TCC CAA TCA TCC C | SEQ ID NO: 16 |
| | 9-F | GGC GTT CTT CTA ACT CAT ACC C | SEQ ID NO: 17 |
| | 9-R | CTC CAT CAC CTA AGG ACT GTT C | SEQ ID NO: 18 |
| | 10-F | GCC TTA TTG TCT TAT TGG TCT | SEQ ID NO: 19 |
| | 10-R | GAA AAA TGG AAT AGC ACC TAA | SEQ ID NO: 20 |
| EAEC | 11-F | GAT GCT GAC GAT TCT GTA TTA | SEQ ID NO: 21 |
| | 11-R | ATA AGT CCT TCT CGA TTG TGT | SEQ ID NO: 22 |
| EHEC | 12-F | GAT AGA TCC AGA GGA AGG GCG | SEQ ID NO: 23 |
| | 12-R | TAC GAC TGA TCC CTG CAA CAC | SEQ ID NO: 24 |
| | 13-F | ACT GTC TGA AAC TGC TCC TGT | SEQ ID NO: 25 |
| | 13-R | GGT TGA CTC TCT TCA TTC ACG | SEQ ID NO: 26 |
| | 14-F | CTC AGG ATG CTA AAC CAG TAG AG | SEQ ID NO: 27 |
| | 14-R | CCG GTA CAA GCA GGA TTA CAA C | SEQ ID NO: 28 |
| EPEC | 15-F | TAG TGG ATT GGA CTC AAC GAT | SEQ ID NO: 29 |
| | 15-R | TAT TAA CAC CGT AGC CTT TCG | SEQ ID NO: 30 |
| EIEC | 16-F | ACG AGT CAA CTT TTA GCG AAG GG | SEQ ID NO: 31 |
| | 16-R | CTC TAT TTC CAA TCG CGT CAG AAC | SEQ ID NO: 32 |
| | 17-F | TCC TGC TTA GAT GAT GGA GG | SEQ ID NO: 33 |
| | 17-R | CCA AAA GGA AGT GTC TGC TC | SEQ ID NO: 34 |
| ETEC | 18-F | TGA CGG ATA TGT TTC CAC TTC | SEQ ID NO: 35 |
| | 18-R | GTA TTC CAC CTA ACG CAG AAA | SEQ ID NO: 36 |
| Listeria | 19-F | CTG GTG ATA CTC TTT GGG GTA | SEQ ID NO: 37 |
| monocytogenes | | | |
| | 19-R | AGC CGT TAG ATT CGG TTG TTT C | SEQ ID NO: 38 |
| | 20-F | GTG ACG AAG TAG AAG TTA TCG | SEQ ID NO: 39 |
| | 20-R | AGT TAG TGT GTG GAG TAA TCG | SEQ ID NO: 40 |
| | 21-F | TTG ATG GTG CTG TTG CGG TTC | SEQ ID NO: 41 |
| | 21-R | TGG GAG TTG GAT TGG GTG C | SEQ ID NO: 42 |
| Salmonella | 22-F | CGC ACT GAA TAT CGT ACT GGA | SEQ ID NO: 43 |
| typhimurium | | | |
| | 22-R | CGA TAA TTT CAC CGG CAT CG | SEQ ID NO: 44 |
| | 23-F | GCT GAG TAA CCA ACA AGA TAA | SEQ ID NO: 45 |
| | 23-R | AGT AAA CGC TGT TCA TAG GTC | SEQ ID NO: 46 |
| Staphylococcus | 24-F | GCA GCT TGC TTA CTT ACT GCT | SEQ ID NO: 47 |
| aureus | | | |
| | 24-R | TAC CTG TAA TCT CGC CAT CAT | SEQ ID NO: 48 |
| | 25-F | ATT CAT TGC CCT AAC GTG GAC | SEQ ID NO: 49 |
| | 25-R | GCT GTA AAA ATT GAT CGT GAC TCT C | SEQ ID NO: 50 |
| | 26-F | GTA TGG TGG TGT AAC TGA GC | SEQ ID NO: 51 |
| | 26-R | CCG TTT CAT AAG GCG AGT TG | SEQ ID NO: 52 |
| | 27-F | CTG CTA TTT TTC ATC CAA AGA | SEQ ID NO: 53 |
| | 27-R | TTC TTA TCA GTT TGC ACT TCA | SEQ ID NO: 54 |
| | 28-F | TGT CAC TCC ACA CGA AGG TA | SEQ ID NO: 55 |
| | 28-R | TGC AAA TAG CGC CTT GCT TG | SEQ ID NO: 56 |
| Vibrio cholerae | 29-F | GCC AAG AGG ACA GAG TGA GTA | SEQ ID NO: 57 |
| | 29-R | ATG AGG ACT GTA TGC CCC TA | SEQ ID NO: 58 |
| | 30-F | TAA GAC CAA CAG CAA CCG CCC | SEQ ID NO: 59 |
| | 30-R | ACT CGA CTG GCG TAA CCA AAA GG | SEQ ID NO: 60 |
| | 31-F | GTT TGT ATG TGC GAG CGG GTG | SEQ ID NO: 61 |
| | 31-R | GTG AAT GTC AGC GCC ACC AAC | SEQ ID NO: 62 |
| Vibrio | 32-F | GCA CTG TCT CAT TTC CCA GAG | SEQ ID NO: 63 |
| parahaemolyticus | | | |
| | 32-R | CGC TTC TTG GTC AGA AAC CAG | SEQ ID NO: 64 |
| | 33-F | ACC TGT GGC TTC TGC TGT G | SEQ ID NO: 65 |
| | 33-R | CCA GTT GTT GAT TTG CGG GTG | SEQ ID NO: 66 |
| | 34-F | TCC ATC TGT CCC TTT TCC TGC C | SEQ ID NO: 67 |
| | 34-R | CAG CCA TTT AGT ACC TGA CGT TGT G | SEQ ID NO: 68 |
| | 35-F | GCG AGC GAT CCT TGT TTG GAC | SEQ ID NO: 69 |
| | 35-R | GCG GTG AGT TGC TGT TGT TGG | SEQ ID NO: 70 |
| Vibrio vulnificus | 36-F | CTC TGC CTA GAT GTT TAT GG | SEQ ID NO: 71 |
| | 36-R | CAA TAC CAT TTC TGT GCT AAG | SEQ ID NO: 72 |
| | 37-F | AGC ACA TCT CTA TTC CTT CTC | SEQ ID NO: 73 |
| | 37-R | TAG CGT TGC TTC TTC AGT AA | SEQ ID NO: 74 |
| Yersinia | 38-F | TGG GGC CAT CTT TCC GCA TTA | SEQ ID NO: 75 |
| enterocolitica | | | |
| | 38-R | TAC CCT GCA CCA AGC ATC CAA | SEQ ID NO: 76 |
| | 39-F | AAC GGG GCA TCT GGT TCT CTC | SEQ ID NO: 77 |
| | 39-R | TGG TGG TGT CAG GAA AGG GAC | SEQ ID NO: 78 |

### Example 2. Identification of Detectability of Primer

PCR was performed to identify whether the prepared primers specifically detect respective target bacteria.

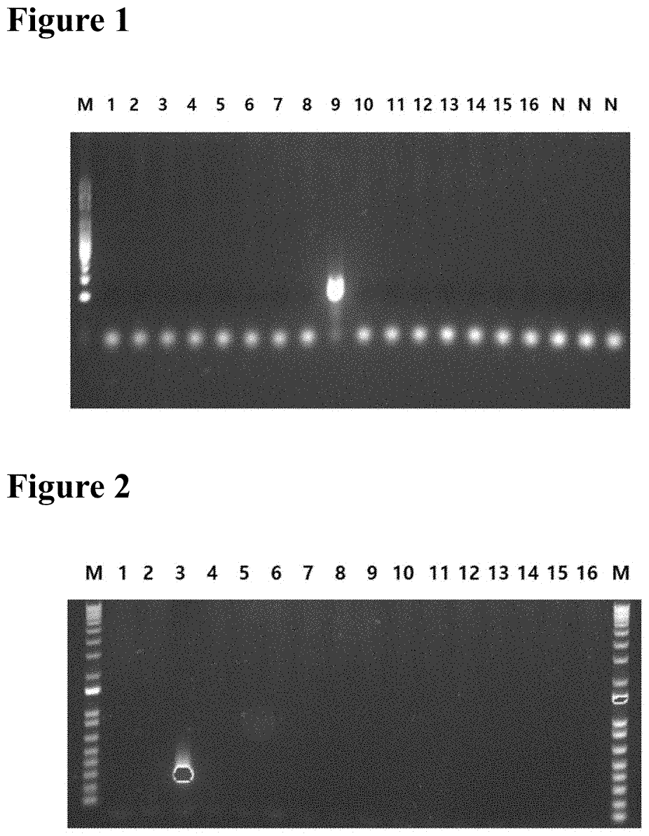

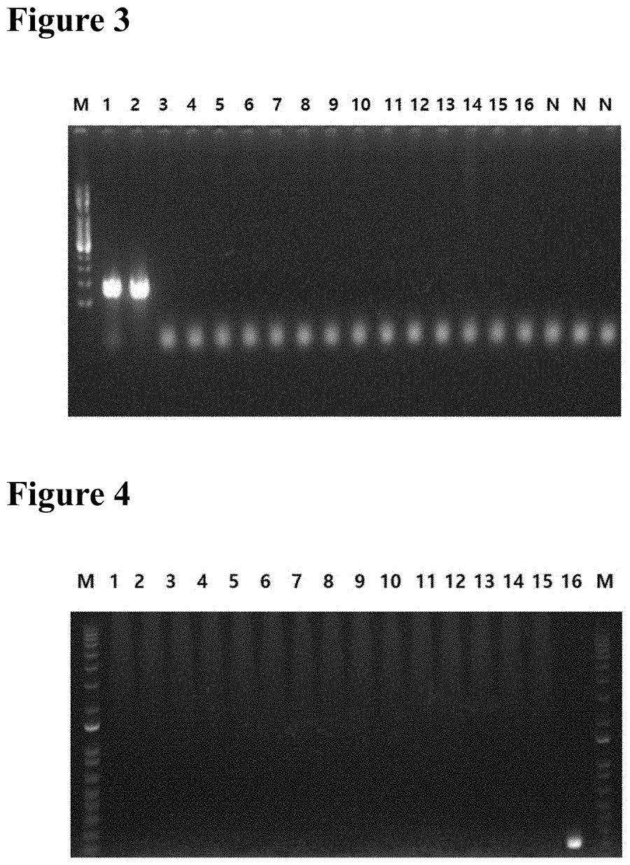

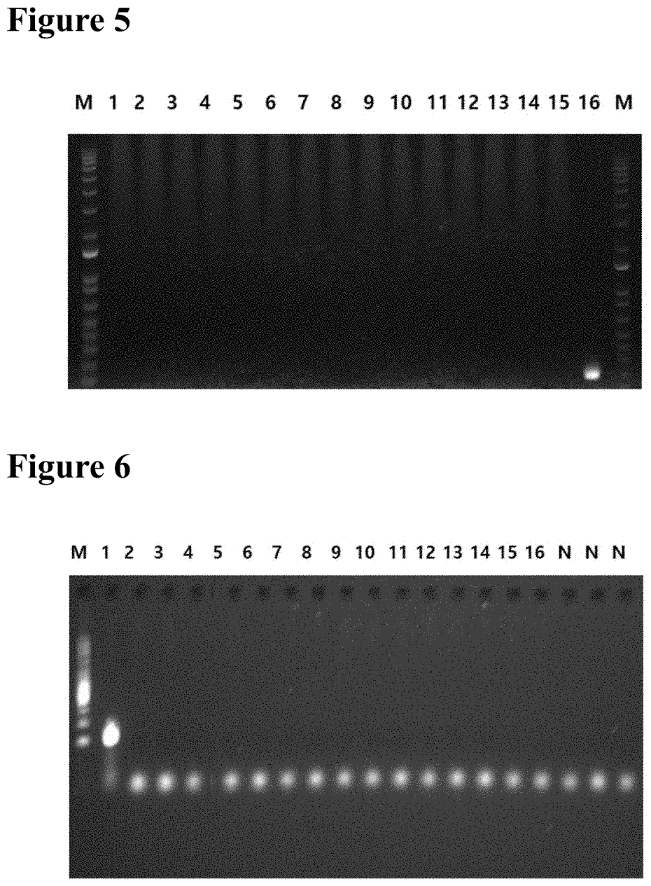

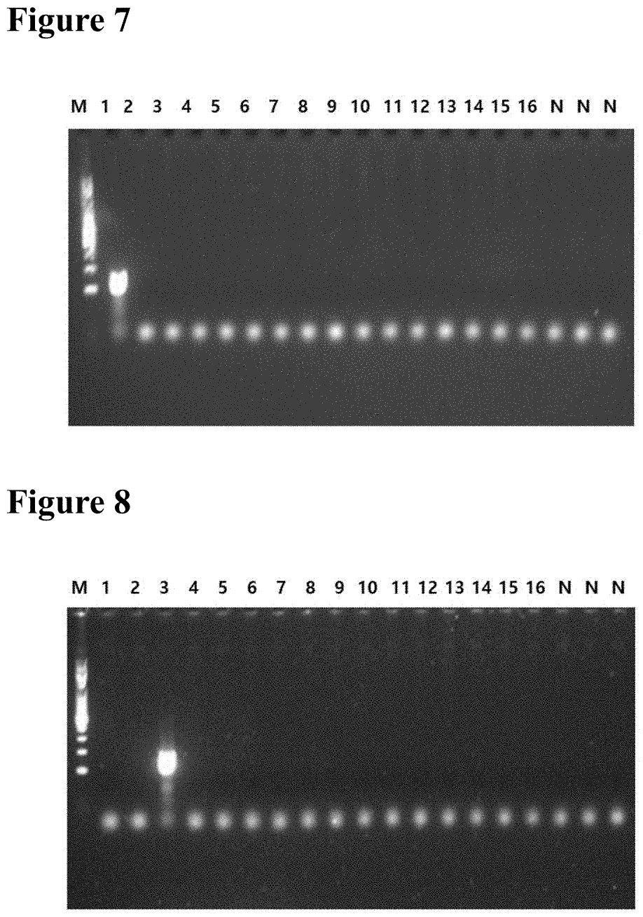

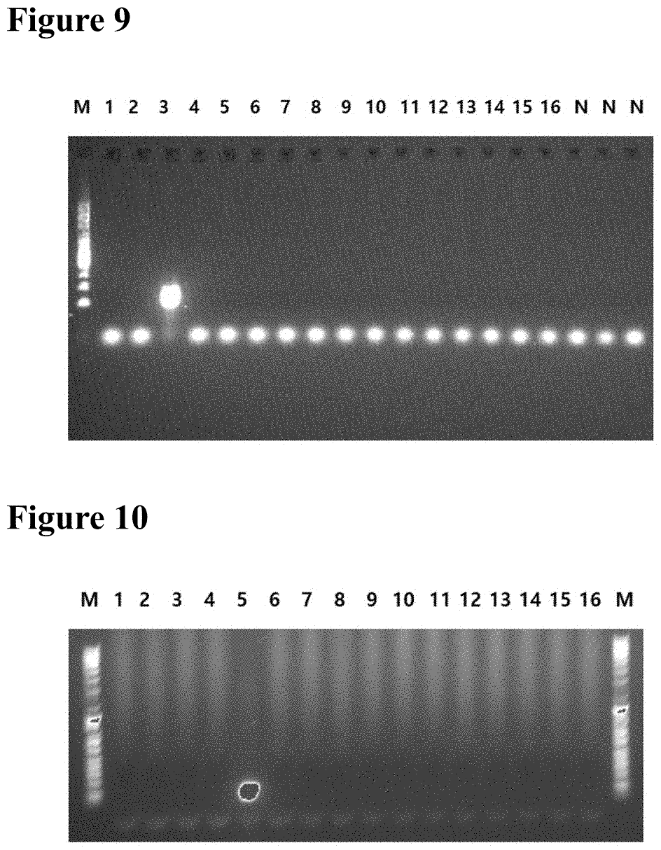

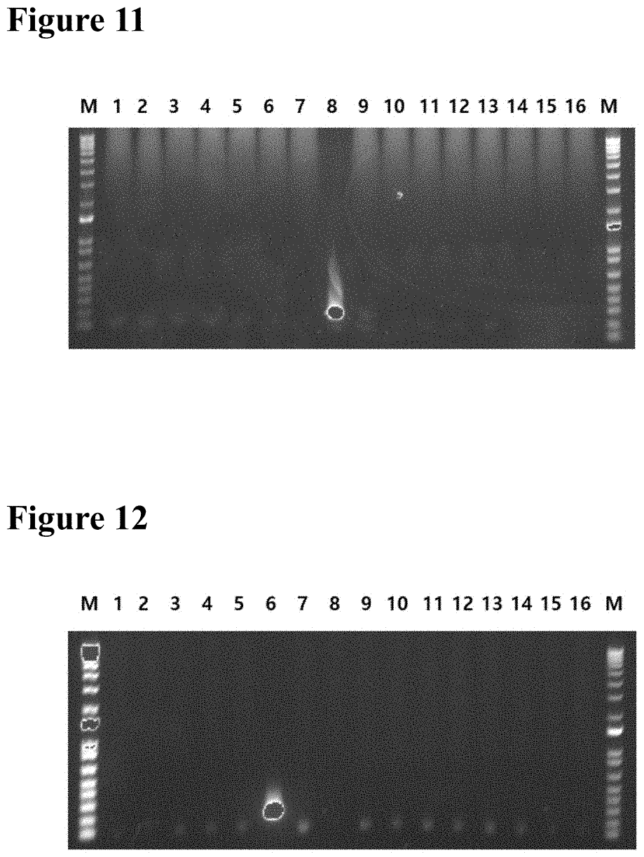

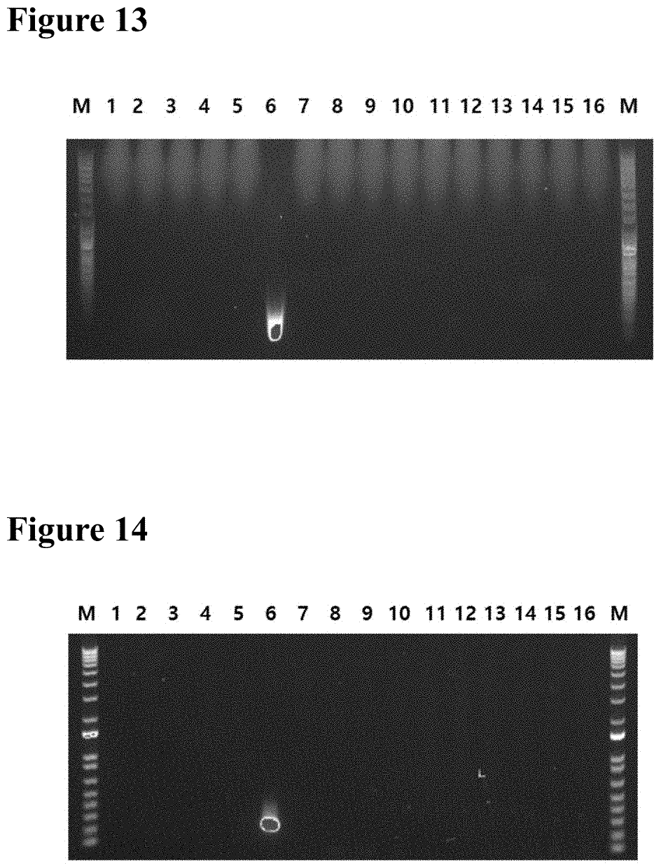

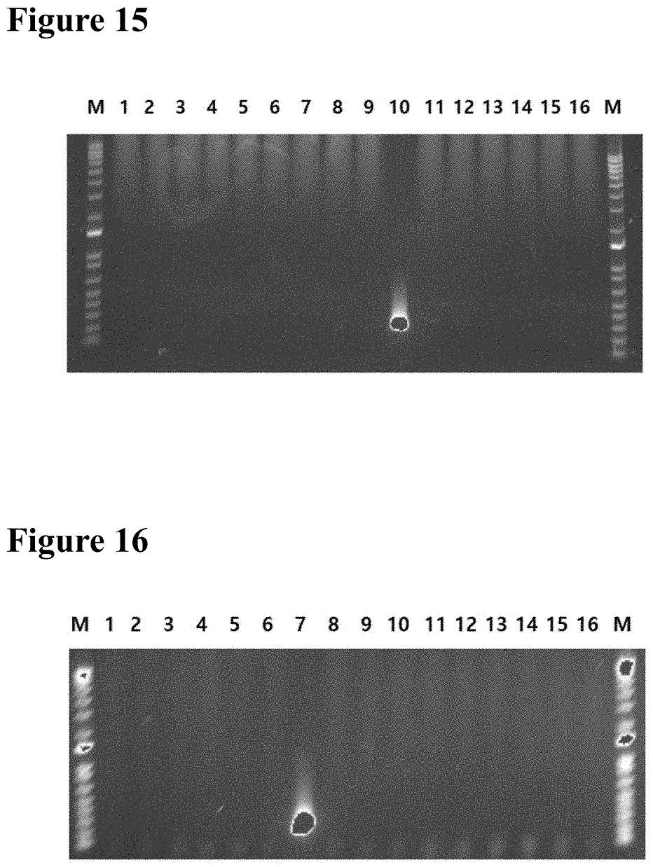

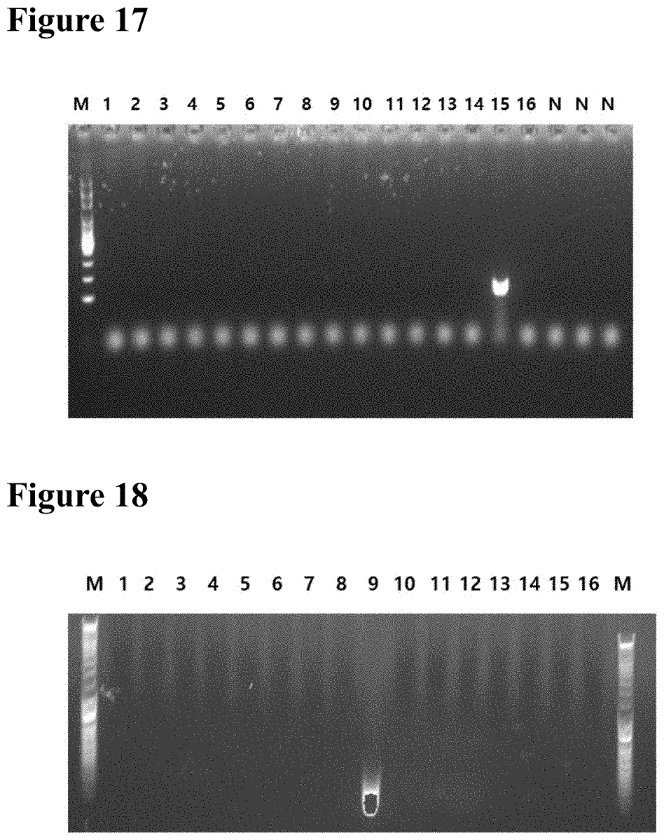

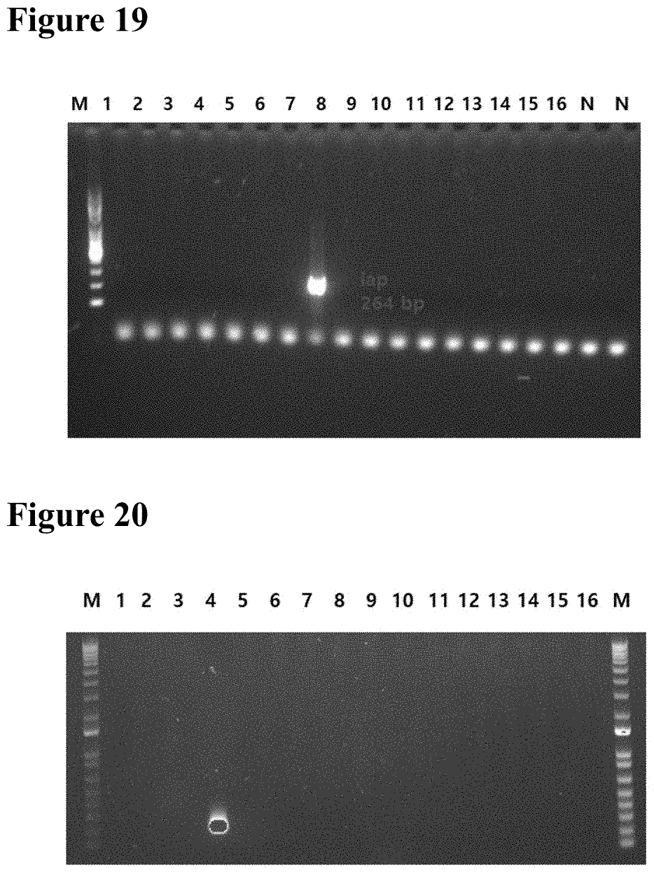

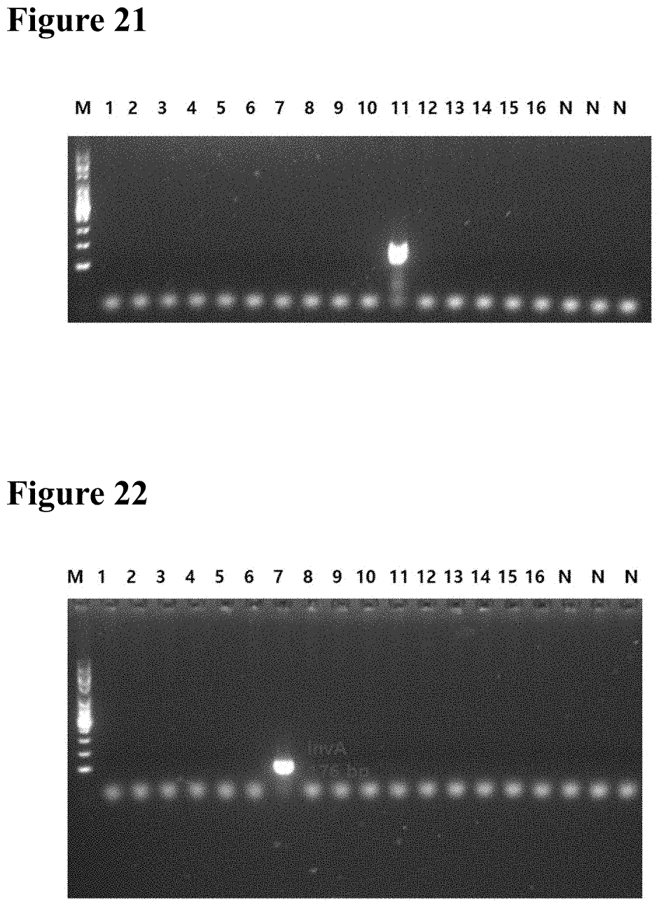

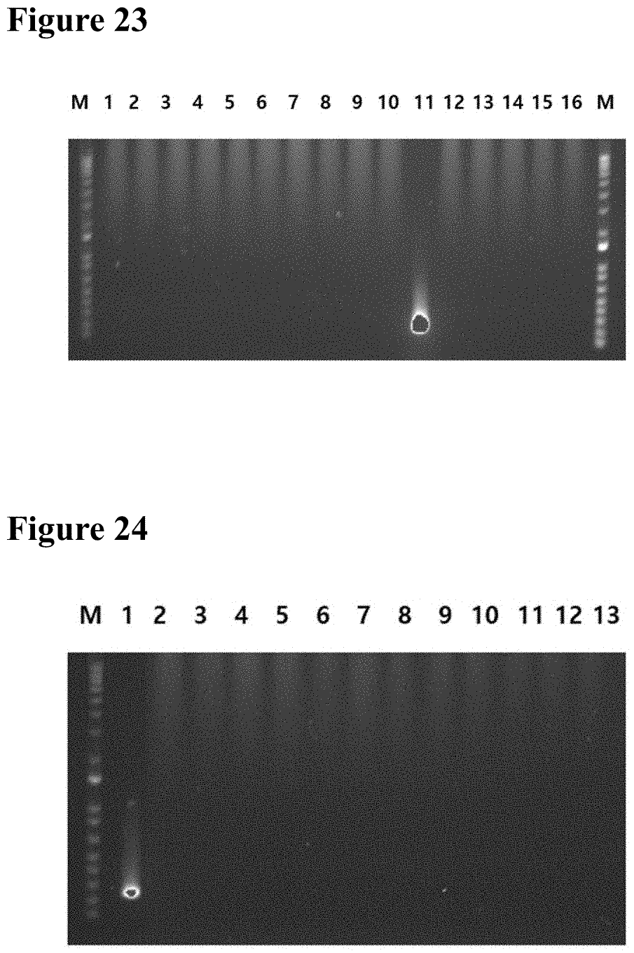

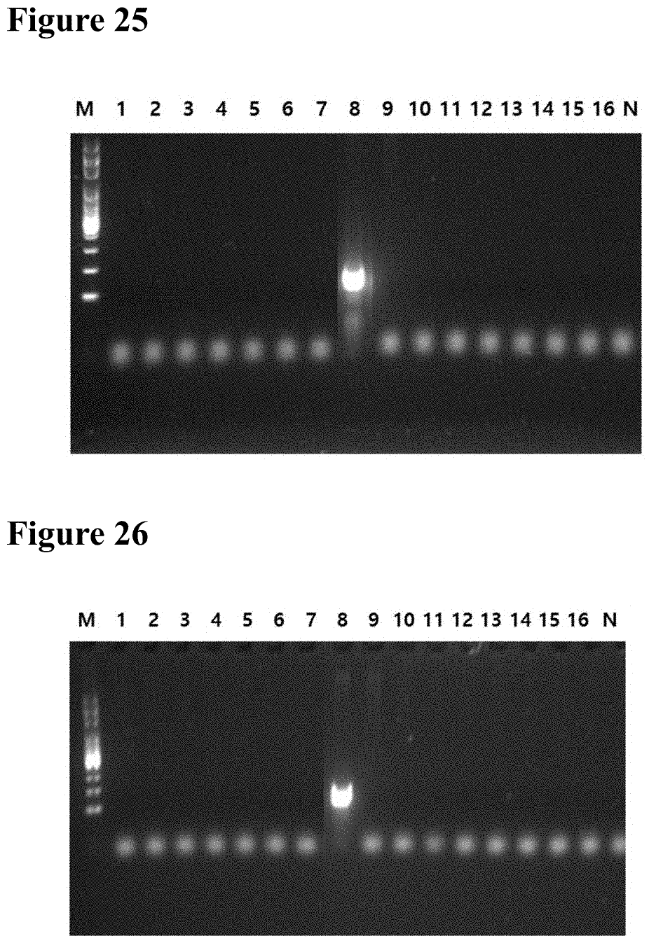

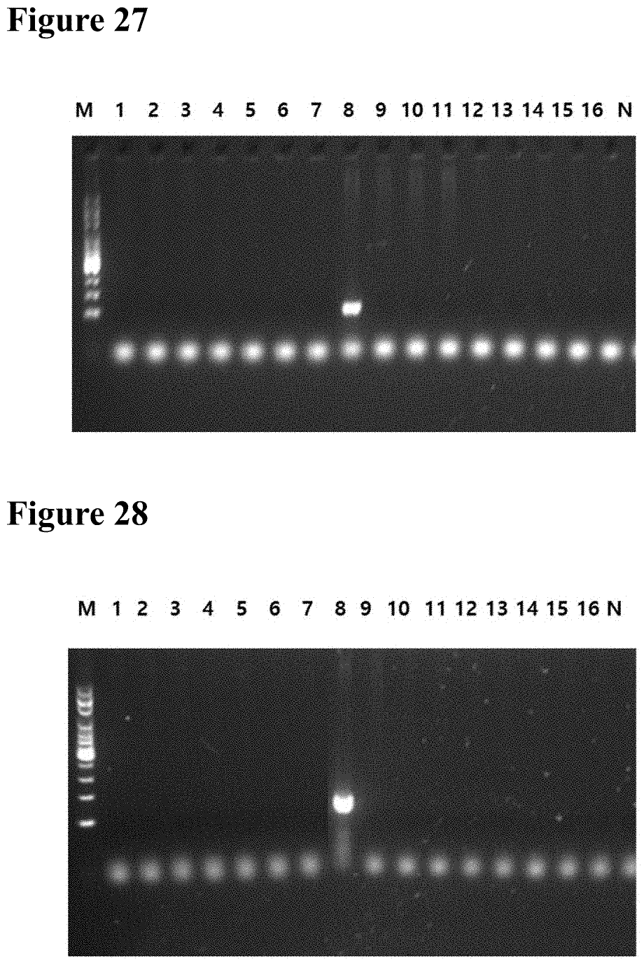

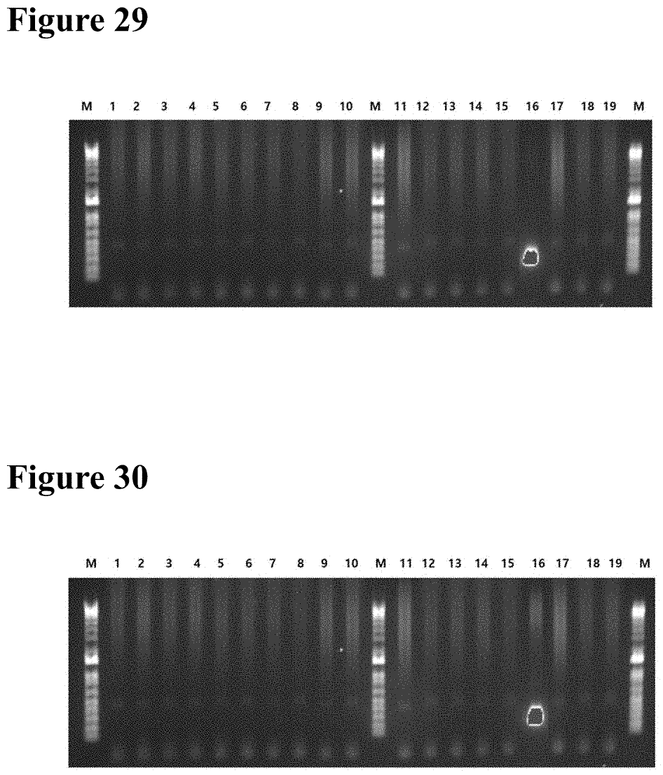

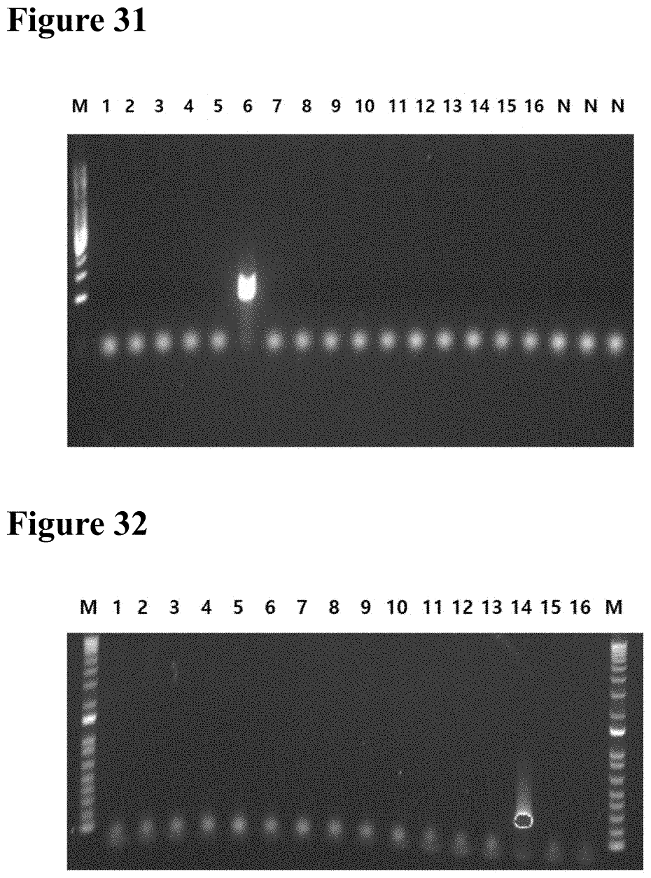

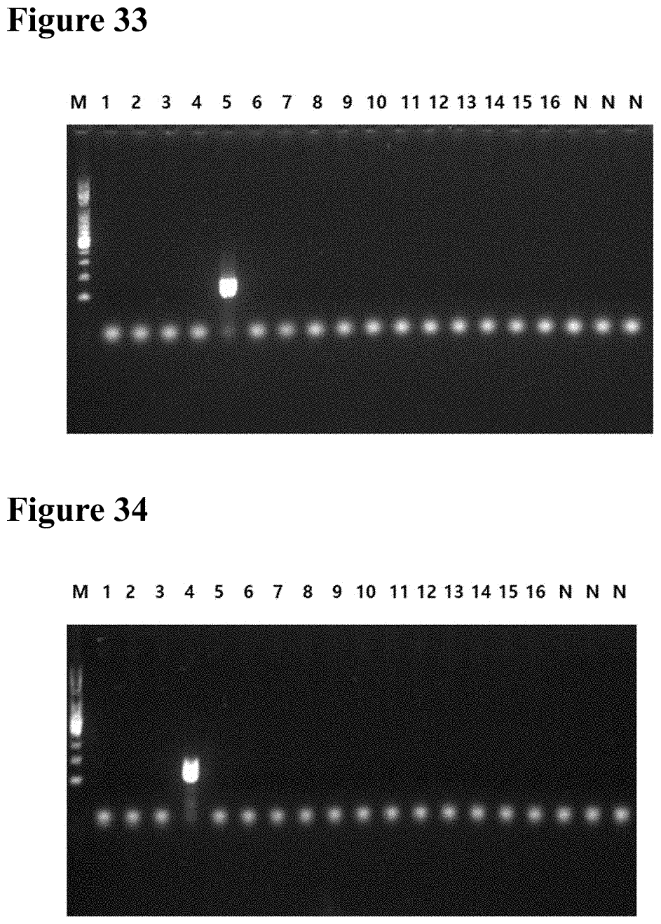

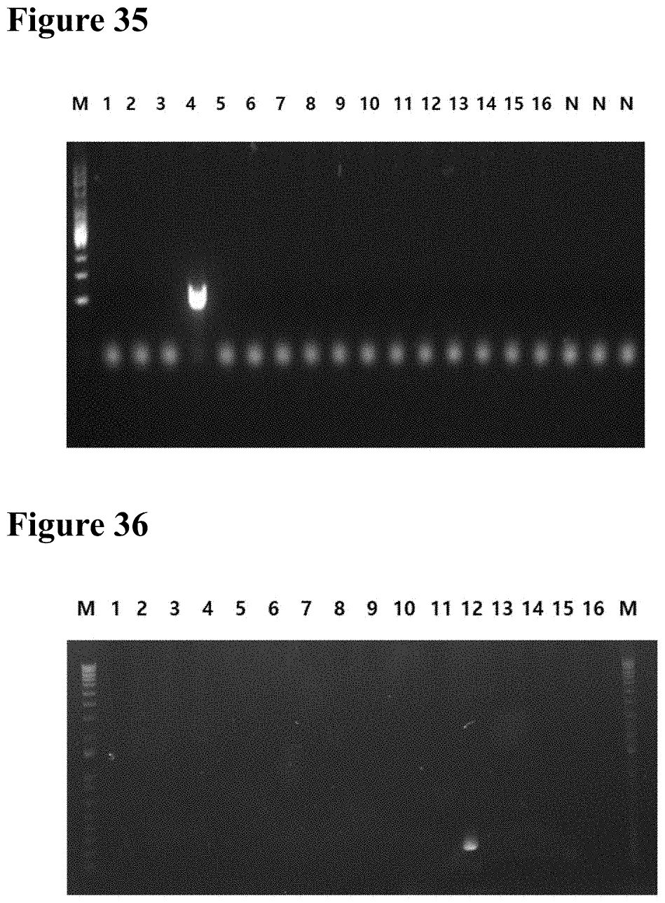

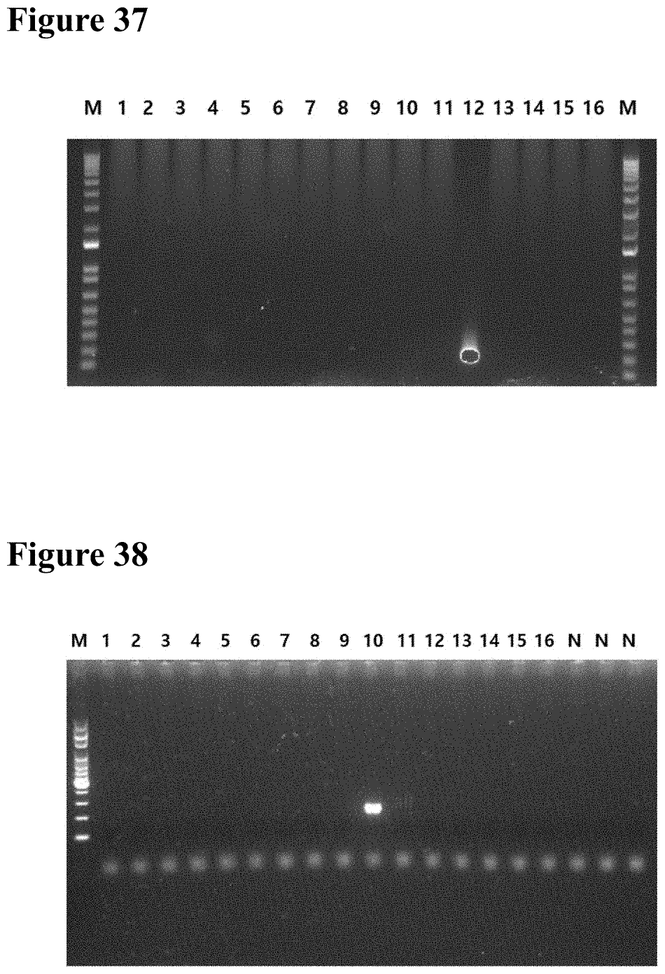

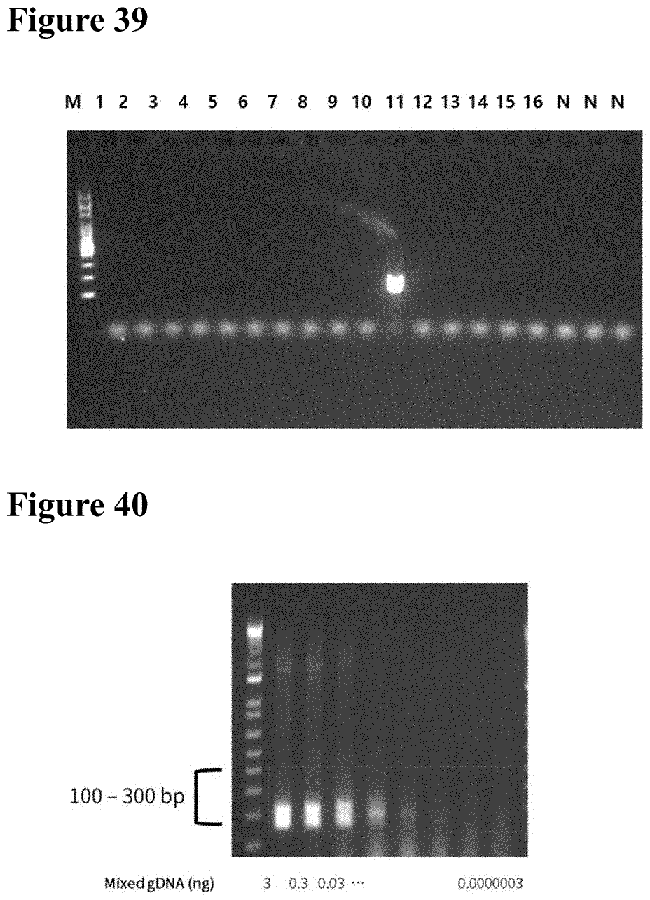

Specifically, using each gene of the 16 types of food poisoning bacteria as a template, whether each of the 1^{st} to 39^{th} primer sets specifically detects certain target bacteria was identified. The strains used were Bacillus cereus (Sanigen, Korea), Campylobacter coli (Sanigen, Korea), Campylobacter jejuni (Sanigen, Korea), Clostridium perfringens (FORC_25), enteroaggregative Escherichia coli (EAEC)(NCCP14039), enterohemorrhagic Escherichia coli (EHEC) (Sanigen, Korea), enteropathogenic Escherichia coli (EPEC) (Sanigen, Korea), enteroinvasive Escherichia coli (EIEC) (Sanigen, Korea), enterotoxigenic Escherichia coli (ETEC) (Sanigen, Korea), Listeria monocytogenes (Sanigen, Korea), Salmonella typhimurium (Sanigen, Korea), Staphylococcus aureus (ATCC25929, ATCC23235, CCARM3089, Newman), Vibrio cholerae (Sanigen, Korea), Vibrio parahaemolyticus (Sanigen, Korea), Vibrio vulnificus (Sanigen, Korea), and Yersinia enterocolitica (Sanigen, Korea). The gene templates were prepared by using Nucleospin® Microbial DNA kit (Macherey-Nagel) in accordance with the manufacturer's protocol. 1 μl of template DNA, 0.1 μl of a forward primer, 0.1 μl of a reverse primer, 10 μl of master mix (Biofact), and 8.8 μl of distilled water were mixed to obtain a PCR reactant. With the PCR reactant, PCR was performed under the conditions described in Table 2 below, and the obtained PCR product was confirmed by using an agarose gel. The results of the agarose gel electrophoresis are shown in FIGS. 1 to 39.

TABLE 2

| Step | Temperature | Time | | Cycle |
|---|---|---|---|---|
| Pre-denaturation | 95° C. | 4 | minutes | 1 time |
| Denaturation | 95° C. | 30 | seconds | 35 times |
| Annealing | 60° C. | 30 | seconds | |
| Extension | 72° C. | 30 | seconds | |
| Post-extension | 72° C. | 5 | minutes | 1 time |

As shown in FIGS. 1 to 39, the 1^{st} to 39^{th} primer sets specifically detected respective target food poisoning bacteria.

### Example 3. Detection of Food Poisoning Bacteria by Using NGS

### 3-1. Amplification of Target Sequence

Both the primers described in Table 1 and the genes of the 16 types of food poisoning bacteria were mixed, and PCR was performed to amplify the target sequences. First, a template DNA mixture was prepared by mixing the DNAs of the 16 strains to be 3 ng, and a forward or reverse primer was prepared by mixing the primers listed in Table 1 to be 10 pmol. Thereafter, 2.5 μl of template DNA mixture, 5 μl of a forward primer mixture, 5 μl of a reverse primer mixture, and 12.5 μl of HIFI KAPA hyperflus master mix (Roche) were mixed to obtain a PCR reactant. With the PCR reactant, PCR was performed under the conditions described in Table 2 below, and the obtained PCR product was confirmed by using an agarose gel. The results of the agarose gel electrophoresis are shown in FIG. 40.

As shown in FIG. 40, PCR products having a size in the range of 150 to 250 bp were prepared as a result of the amplification by the 39 primer sets described in Table 1.

### 3-2. Preparation of Library

Using the thus-obtained PCR products, a library was prepared as follows.

First, 50 μl of a sample purification bead (SPB, Illumina) and 25 μl of the PCR products were mixed well on the tube and incubated for 5 minutes at room temperature. After the incubation, the tube was transferred to the magnetic stand and incubated further for 3 minutes until the liquid was clear, and the supernatant was removed. The remained bead was washed by adding 200 μl of 80% ethanol. The mixture was incubated on the magnetic stand for 30 seconds. After then, the supernatant of ethanol was removed. This process was repeated twice to wash the beads and completely remove the ethanol. 62.5 μl of a resuspension buffer (Illumina) and the washed bead were mixed, and the mixture was incubated for 2 minutes. The mixture was transferred to the magnetic stand and incubated for a further 3 minutes, and then 60 μl of the supernatant was taken and transferred to a new tube. 40 μl of an end repair mix2 (Illumina) was added to the new tube, and the mixture was reacted for 30 minutes at 30° C. to repair incomplete ends. After the reaction was finished, the reactant and 160 μl of SPB were mixed well, and the mixture was incubated for 5 minutes at room temperature. The mixture was transferred to the magnetic stand and incubated for a further 5 minutes, and the supernatant was removed. Thereafter, the bead was washed by ethanol, a resuspension buffer was added to the washed bead, and 17.5 μl of the supernatant was taken. The 17.5 μl of the supernatant and 12.5 μl of A-tailing mix (Illumina) was mixed and reacted for 30 minutes at 97° C. and for 5 minutes at 72° C. 30 μl of the resulting reactant, 2.5 μl of a ligation mix (Illumina), TruSeq DNA CD indexes (Illumina) and 2.5 μl of a resuspension buffer were mixed. The mixture was reacted at 30° C. for 10 minutes, and 5 μl of a stop ligation buffer (Illumina) was added to terminate the reaction. 42.5 μl of the reaction product, 46 μl of sample purification bead, and 46 μl of deionized water were mixed well, and the mixture was incubated for 5 minutes at room temperature. Only the supernatant was taken and placed in a new tube. 105 μl of sample purification bead was added, and the mixture was incubated at room temperature for 5 minutes. Thereafter, the bead was washed with ethanol as described above, and the process of adding and reacting a resuspension buffer to the washed bead was repeated twice to obtain 25 μl of a library.

### 3-3. Library Amplification

The thus-prepared library was amplified as follows.

Specifically, 25 μl of the library, 20 μl of enhanced PCR mix (Illumina), 5 μl of the primer mixture prepared in Example 3-1 were mixed to obtain a PCR reactant. With the PCR reactant, PCR was performed under the conditions as described in Table 3 below.

TABLE 3

| Step | Temperature | Time | | Cycle |
|---|---|---|---|---|
| Pre-denaturation | 98° C. | 3 | minutes | 1 time |
| Denaturation | 98° C. | 20 | seconds | 12 times |
| Annealing | 60° C. | 15 | seconds | |
| Extension | 72° C. | 30 | seconds | |
| Post-extension | 72° C. | 5 | minutes | 1 time |

The thus-obtained PCR product and 50 μl of sample purification bead were mixed, the mixture was incubated and washed as described above, and a resuspension buffer was added to obtain 30 μl of a PCR product.

### 3-4. Measurement of Concentration of Final Product and Performance of NGS

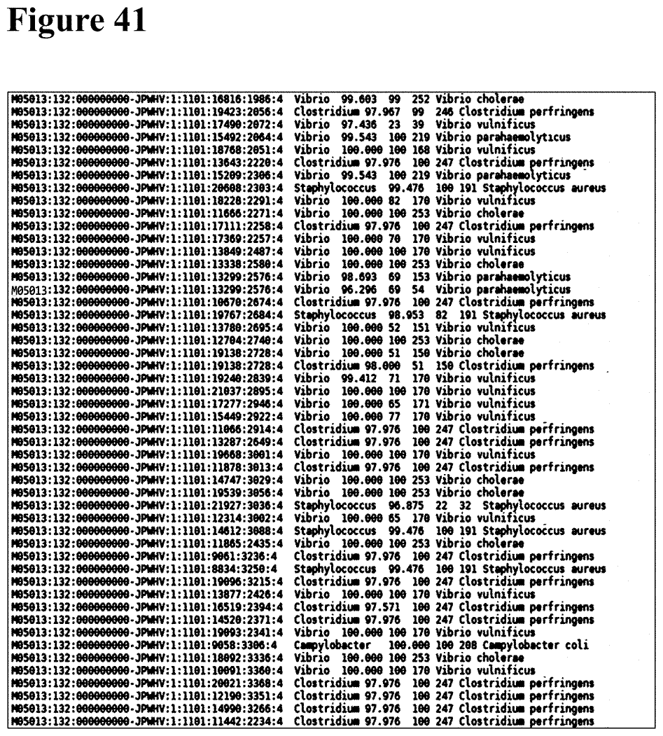

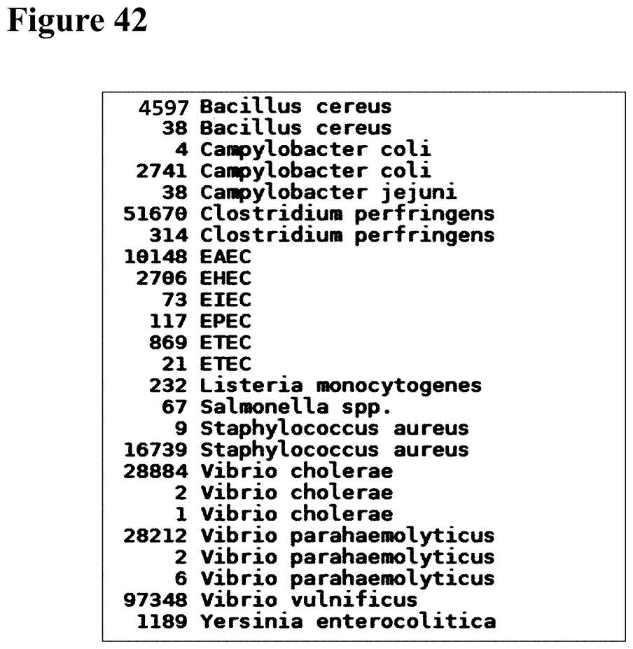

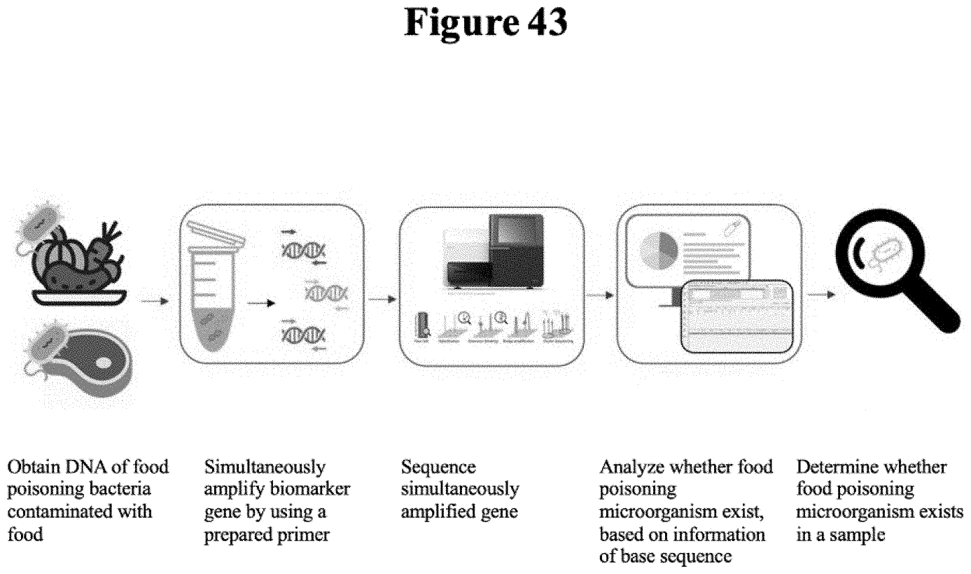

The concentration of the PCR product obtained above was measured by using a bio-analyzer, and the concentration was adjusted to be 4 nM. 4 nM of the PCR product was diluted to 7 pM, and the diluted PCRproduct and a phiX solution (Illumina) were mixed with a volume ratio of 7:3. With the mixture, NGS was performed by Miseq NGS equipment according to the manufacturer's protocol. Data obtained through NGS performance was analyzed by using Trimmomatic, Pandaseq, and BLAST. Specifically, the read values obtained by the Miseq NGS equipment was filtered based on the phred score of 15, and the values were summed by read unit. On the other hand, by a BLAST analysis of the base sequence of the target region amplified by the primer was filtered based on 95% homology. In this process, the final result was achieved by counting the number of matching read values. A portion of the analysis data is shown in FIG. 41, the result of combining the number of target sites detected is shown in FIG. 42, and analysis data with respect to the strains is shown in Table 4.

TABLE 4

| | Target | Amplicon | | | | |
|---|---|---|---|---|---|---|
| Strains | location | size (bp) | 3.E+00 | 3.E−01 | 3.E−02 | 3.E−03 |
| Bacillus cereus | 1 | 175 | 857 | 361 | 490 | 296 |
| | 2 | 229 | 6426 | 3881 | 2373 | 2177 |
| Campylobacter spp. | 3 | 191 | 608 | 194 | 290 | 173 |
| Campylobacter coli | 4 | 203 | 6179 | 6389 | 11298 | 13502 |
| | 5 | 208 | 8514 | 7689 | 7722 | 9281 |
| Campylobacter jejuni | 6 | 184 | 2390 | 265 | 129 | 38 |
| | 7 | 163 | 8682 | 7046 | 11693 | 8354 |
| Clostridium perfringens | 8 | 247 | 10990 | 9645 | 8051 | 14150 |
| | 9 | 168 | 3820 | 2427 | 11369 | 9258 |
| | 10 | 178 | 2890 | 3322 | 9173 | 13574 |
| EAEC | 11 | 187 | 21964 | 21054 | 24069 | 38173 |
| EHEC | 12 | 209 | 26 | 17 | 7 | 2 |
| | 13 | 255 | 10858 | 7945 | 10576 | 8052 |
| | 14 | 154 | 1967 | 832 | 673 | 413 |
| EPEC | 15 | 233 | 12399 | 6306 | 5858 | 3006 |
| EIEC | 16 | 234 | 8703 | 4226 | 1938 | 3196 |
| | 17 | 173 | 635 | 201 | 84 | 36 |
| ETEC | 18 | 191 | 2640 | 3691 | 8497 | 8303 |
| Listeria monocytogenes | 19 | 264 | 5269 | 1296 | 1055 | 743 |
| | 20 | 198 | 2488 | 683 | 105 | 50 |
| | 21 | 200 | 26 | 1 | 0 | 1 |
| Salmonella typhimurium | 22 | 176 | 1620 | 1092 | 2272 | 714 |
| | 23 | 186 | 4893 | 2172 | 549 | 336 |
| Staphylococcus aureus | 24 | 214 | 914 | 2172 | 3478 | 518 |
| | 25 | 191 | 14317 | 12622 | 22756 | 54297 |
| | 26 | 212 | 1480 | 1539 | 838 | 818 |
| | 27 | 180 | 1203 | 2015 | 831 | 893 |
| | 28 | 162 | 2054 | 768 | 10 | 278 |
| Vibrio cholerae | 29 | 253 | 5067 | 3656 | 5803 | 5795 |
| | 30 | 209 | 2955 | 987 | 325 | 223 |
| | 31 | 175 | 319 | 90 | 21 | 3 |
| Vibrio parahaemolyticus | 32 | 219 | 3832 | 2287 | 9705 | 7423 |
| | 33 | 178 | 2814 | 837 | 312 | 79 |
| | 34 | 190 | 1499 | 126 | 4 | 13 |
| | 35 | 144 | 65 | 18 | 23 | 31 |
| Vibrio vulnificus | 36 | 218 | 4557 | 851 | 52 | 103 |
| | 37 | 170 | 11946 | 13190 | 31663 | 42789 |
| Yersinia enterocolitica | 38 | 202 | 20 | 8 | 1 | 2 |
| | 39 | 190 | 4348 | 3257 | 3187 | 1204 |

As shown in Table 4, 16 types of food poisoning bacteria were detected at once by the NGS method by using the primers according to the present invention.

1. A primer set for detecting food poisoning bacteria, the primer set comprising at least one selected from the group consisting of:
the 1^{st} primer pair consisting of a base sequence represented by SEQ ID NO: 1 or 2;
the 2^{nd} primer pair consisting of a base sequence represented by SEQ ID NO: 3 or 4;
the 3^{rd} primer pair consisting of a base sequence represented by SEQ ID NO: 5 or 6;
the 4^{th} primer pair consisting of a base sequence represented by SEQ ID NO: 7 or 8;
the 5^{th} primer pair consisting of a base sequence represented by SEQ ID NO: 9 or 10;
the 6^{th} primer pair consisting of a base sequence represented by SEQ ID NO: 11 or 12;
the 7^{th} primer pair consisting of a base sequence represented by SEQ ID NO: 13 or 14;
the 8^{th} primer pair consisting of a base sequence represented by SEQ ID NO: 15 or 16;
the 9^{th} primer pair consisting of a base sequence represented by SEQ ID NO: 17 or 18;
the 10^{th} primer pair consisting of a base sequence represented by SEQ ID NO: 19 or 20;
the 11^{th} primer pair consisting of a base sequence represented by SEQ ID NO: 21 or 22;
the 12^{th} primer pair consisting of a base sequence represented by SEQ ID NO: 23 or 24;
the 13^{th} primer pair consisting of a base sequence represented by SEQ ID NO: 25 or 26;
the 14^{th} primer pair consisting of a base sequence represented by SEQ ID NO: 27 or 28;
the 15^{th} primer pair consisting of a base sequence represented by SEQ ID NO: 29 or 30;
the 16^{th} primer pair consisting of a base sequence represented by SEQ ID NO: 31 or 32;
the 17^{th} primer pair consisting of a base sequence represented by SEQ ID NO: 33 or 34;
the 18^{th} primer pair consisting of a base sequence represented by SEQ ID NO: 35 or 36;
the 19^{th} primer pair consisting of a base sequence represented by SEQ ID NO: 37 or 38;
the 20^{th} primer pair consisting of a base sequence represented by SEQ ID NO: 39 or 40;
the 21^{st} primer pair consisting of a base sequence represented by SEQ ID NO: 41 or 42;
the 22^{nd} primer pair consisting of a base sequence represented by SEQ ID NO: 43 or 44;
the 23^{rd} primer pair consisting of a base sequence represented by SEQ ID NO: 45 or 46;
the 24^{th} primer pair consisting of a base sequence represented by SEQ ID NO: 47 or 48;
the 25^{th} primer pair consisting of a base sequence represented by SEQ ID NO: 49 or 50;
the 26^{th} primer pair consisting of a base sequence represented by SEQ ID NO: 51 or 52;
the 27^{th} primer pair consisting of a base sequence represented by SEQ ID NO: 53 or 54;
the 28^{th} primer pair consisting of a base sequence represented by SEQ ID NO: 55 or 56;
the 29^{th} primer pair consisting of a base sequence represented by SEQ ID NO: 57 or 58;
the 30^{th} primer pair consisting of a base sequence represented by SEQ ID NO: 59 or 60;
the 3 Pt primer pair consisting of a base sequence represented by SEQ ID NO: 61 or 62;
the 32^{nd} primer pair consisting of a base sequence represented by SEQ ID NO: 63 or 64;
the 33^{rd} primer pair consisting of a base sequence represented by SEQ ID NO: 65 or 66;
the 34^{th} primer pair consisting of a base sequence represented by SEQ ID NO: 67 or 68;
the 35^{th} primer pair consisting of a base sequence represented by SEQ ID NO: 69 or 70;
the 36^{th} primer pair consisting of a base sequence represented by SEQ ID NO: 71 or 72;
the 37^{th} primer pair consisting of a base sequence represented by SEQ ID NO: 73 or 74;
the 38^{th} primer pair consisting of a base sequence represented by SEQ ID NO: 75 or 76; and
the 39^{th} primer pair consisting of a base sequence represented by SEQ ID NO: 77 or 78.

2. The primer set of claim 1 comprising:
a) at least one primer pair selected from the group consisting of the 1^{st} primer pair consisting of a base sequence represented by SEQ ID NO: 1 or 2 and the 2^{nd} primer pair consisting of a base sequence represented by SEQ ID NO: 3 or 4;
b) the 3^{rd} primer pair consisting of a base sequence represented by SEQ ID NO: 5 or 6;
c) at least one primer pair selected from the group consisting of the 4^{th} primer pair consisting of a base sequence represented by SEQ ID NO: 7 or 8 and the 5^{th} primer pair consisting of a base sequence represented by SEQ ID NO: 9 or 10;
d) at least one primer pair selected from the group consisting of the 6^{th} primer pair consisting of a base sequence represented by SEQ ID NO: 11 or 12 and the 7^{th} primer pair consisting of a base sequence represented by SEQ ID NO: 13 or 14;
e) at least one primer pair selected from the group consisting of the 8^{th} primer pair consisting of a base sequence represented by SEQ ID NO: 15 or 16, the 9^{th} primer pair consisting of a base sequence represented by SEQ ID NO: 17 or 18, and the 10^{th} primer pair consisting of a base sequence represented by SEQ ID NO: 19 or 20;
f) the 11^{th} primer pair consisting of a base sequence represented by SEQ ID NO: 21 or 22;
g) at least one primer pair selected from the group consisting of the 12^{th} primer pair consisting of a base sequence represented by SEQ ID NO: 23 or 24, the 13^{th} primer pair consisting of a base sequence represented by SEQ ID NO: 25 or 26, and the 14^{th} primer pair consisting of a base sequence represented by SEQ ID NO: 27 or 28;
h) the 15^{th} primer pair consisting of a base sequence represented by SEQ ID NO: 29 or 30;
i) at least one primer pair selected from the group consisting of the 16^{th} primer pair consisting of a base sequence represented by SEQ ID NO: 31 or 32 and the 17^{th} primer pair consisting of a base sequence represented by SEQ ID NO: 33 or 34;
j) the 18^{th} primer pair consisting of a base sequence represented by SEQ ID NO: 35 or 36;
k) at least one primer pair selected from the group consisting of the 19^{th} primer pair consisting of a base sequence represented by SEQ ID NO: 37 or 38, the 20^{th} primer pair consisting of a base sequence represented by SEQ ID NO: 39 or 40, and the 21^{st} primer pair consisting of a base sequence represented by SEQ ID NO: 41 or 42;
l) at least one primer pair selected from the group consisting of the 22^{nd} primer pair consisting of a base sequence represented by SEQ ID NO: 43 or 44 and the 23^{rd} primer pair consisting of a base sequence represented by SEQ ID NO: 45 or 46;
m) at least one primer pair selected from the group consisting of the 24^{th} primer pair consisting of a base sequence represented by SEQ ID NO: 47 or 48, the 25^{th} primer pair consisting of a base sequence represented by SEQ ID NO: 49 or 50, the 26^{th} primer pair consisting of a base sequence represented by SEQ ID NO: 51 or 52, the 27^{th} primer pair consisting of a base sequence represented by SEQ ID NO: 53 or 54, and the 28^{th} primer pair consisting of a base sequence represented by SEQ ID NO: 55 or 56;
n) at least one primer pair selected from the group consisting of the 29^{th} primer pair consisting of a base sequence represented by SEQ ID NO: 57 or 58, the 30^{th} primer pair consisting of a base sequence represented by SEQ ID NO: 59 or 60, and the 31^{st} primer pair consisting of a base sequence represented by SEQ ID NO: 61 or 62;
o) at least one primer pair selected from the group consisting of the 32^{nd} primer pair consisting of a base sequence represented by SEQ ID NO: 63 or 64, the 33^{rd} primer pair consisting of a base sequence represented by SEQ ID NO: 65 or 66, the 34^{th} primer pair consisting of a base sequence represented by SEQ ID NO: 67 or 68, and the 35^{th} primer pair consisting of a base sequence represented by SEQ ID NO: 69 or 70;
p) at least one primer pair selected from the group consisting of the 36^{th} primer pair consisting of a base sequence represented by SEQ ID NO: 71 or 72 and the 37^{th} primer pair consisting of a base sequence represented by SEQ ID NO: 73 or 74; and
q) at least one primer pair selected from the group consisting of the 38^{th} primer pair consisting of a base sequence represented by SEQ ID NO: 75 or 76 and the 39^{th} primer pair consisting of a base sequence represented by SEQ ID NO: 77 or 78.

3. A primer set comprising:
the 1^{st} primer pair consisting of a base sequence represented by SEQ ID NO: 1 or 2;
the 2^{nd} primer pair consisting of a base sequence represented by SEQ ID NO: 3 or 4;
the 3^{rd} primer pair consisting of a base sequence represented by SEQ ID NO: 5 or 6;
the 4^{th} primer pair consisting of a base sequence represented by SEQ ID NO: 7 or 8;
the 5^{th} primer pair consisting of a base sequence represented by SEQ ID NO: 9 or 10;
the 6^{th} primer pair consisting of a base sequence represented by SEQ ID NO: 11 or 12;
the 7^{th} primer pair consisting of a base sequence represented by SEQ ID NO: 13 or 14;
the 8^{th} primer pair consisting of a base sequence represented by SEQ ID NO: 15 or 16;
the 9^{th} primer pair consisting of a base sequence represented by SEQ ID NO: 17 or 18;
the 10^{th} primer pair consisting of a base sequence represented by SEQ ID NO: 19 or 20;
the 11^{th} primer pair consisting of a base sequence represented by SEQ ID NO: 21 or 22;
the 12^{th} primer pair consisting of a base sequence represented by SEQ ID NO: 23 or 24;
the 13^{th} primer pair consisting of a base sequence represented by SEQ ID NO: 25 or 26;
the 14^{th} primer pair consisting of a base sequence represented by SEQ ID NO: 27 or 28;
the 15^{th} primer pair consisting of a base sequence represented by SEQ ID NO: 29 or 30;
the 16^{th} primer pair consisting of a base sequence represented by SEQ ID NO: 31 or 32;
the 17^{th} primer pair consisting of a base sequence represented by SEQ ID NO: 33 or 34;
the 18^{th} primer pair consisting of a base sequence represented by SEQ ID NO: 35 or 36;
the 19^{th} primer pair consisting of a base sequence represented by SEQ ID NO: 37 or 38;
the 20^{th} primer pair consisting of a base sequence represented by SEQ ID NO: 39 or 40;
the 21^{st} primer pair consisting of a base sequence represented by SEQ ID NO: 41 or 42;
the 22^{nd} primer pair consisting of a base sequence represented by SEQ ID NO: 43 or 44;
the 23^{rd} primer pair consisting of a base sequence represented by SEQ ID NO: 45 or 46;
the 24^{th} primer pair consisting of a base sequence represented by SEQ ID NO: 47 or 48;
the 25^{th} primer pair consisting of a base sequence represented by SEQ ID NO: 49 or 50;
the 26^{th} primer pair consisting of a base sequence represented by SEQ ID NO: 51 or 52;
the 27^{th} primer pair consisting of a base sequence represented by SEQ ID NO: 53 or 54;
the 28^{th} primer pair consisting of a base sequence represented by SEQ ID NO: 55 or 56;
the 29^{th} primer pair consisting of a base sequence represented by SEQ ID NO: 57 or 58;
the 30^{th} primer pair consisting of a base sequence represented by SEQ ID NO: 59 or 60;
the 3 Pt primer pair consisting of a base sequence represented by SEQ ID NO: 61 or 62;
the 32^{nd} primer pair consisting of a base sequence represented by SEQ ID NO: 63 or 64;
the 33^{rd} primer pair consisting of a base sequence represented by SEQ ID NO: 65 or 66;
the 34^{th} primer pair consisting of a base sequence represented by SEQ ID NO: 67 or 68;
the 35^{th} primer pair consisting of a base sequence represented by SEQ ID NO: 69 or 70;
the 36^{th} primer pair consisting of a base sequence represented by SEQ ID NO: 71 or 72;
the 37^{th} primer pair consisting of a base sequence represented by SEQ ID NO: 73 or 74;
the 38^{th} primer pair consisting of a base sequence represented by SEQ ID NO: 75 or 76; and
the 39^{th} primer pair consisting of a base sequence represented by SEQ ID NO: 77 or 78.

4. The primer set of claim 1, wherein the detection is performed by next-generation sequencing (NGS).

5. The primer set of claim 1, wherein the food poisoning bacteria is at least one selected from the group consisting of Bacillus, Campylobacter, Clostridium, Escherichia, Listeria, Salmonella, Staphylococcus, Vibrio, and Yersinia.

6. The primer set of claim 1, wherein the food poisoning bacteria is at least one selected from the group consisting of Bacillus cereus, Campylobacter coli, Campylobacter jejuni, Clostridium perfringens, enteroaggregative Escherichia coli (EAEC), enterohemorrhagic Escherichia coli (EHEC), enteropathogenic Escherichia coli (EPEC), enteroinvasive Escherichia coli (EIEC), enterotoxigenic Escherichia coli (ETEC), Listeria monocytogenes, Salmonella typhimurium, Staphylococcus aureus, Vibrio cholerae, Vibrio parahaemolyticus, Vibrio vulnificus and Yersinia enterocolitica.

7. The primer set of claim 1 comprising:
r) at least one primer pair selected from the group consisting of the 1^{st} primer pair consisting of a base sequence represented by SEQ ID NO: 1 or 2 and the 2^{nd} primer pair consisting of a base sequence represented by SEQ ID NO: 3 or 4, the at least one primer pair being capable of detecting Bacillus cereus;
s) the 3^{rd} primer pair consisting of a base sequence represented by SEQ ID NO: 5 or 6, the 3^{rd} primer pair being capable of detecting Campylobacter;
t) at least one primer pair selected from the group consisting of the 4^{th} primer pair consisting of a base sequence represented by SEQ ID NO: 7 or 8 and the 5^{th} primer pair consisting of a base sequence represented by SEQ ID NO: 9 or 10, the at least one primer pair being capable of detecting Campylobacter coli;
u) at least one primer pair selected from the group consisting of the 6^{th} primer pair consisting of a base sequence represented by SEQ ID NO: 11 or 12 and the 7^{th} primer pair consisting of a base sequence represented by SEQ ID NO: 13 or 14, the at least one primer pair being capable of detecting Campylobacter jejuni;
v) at least one primer pair selected from the group consisting of the 8^{th} primer pair consisting of a base sequence represented by SEQ ID NO: 15 or 16, the 9^{th} primer pair consisting of a base sequence represented by SEQ ID NO: 17 or 18, and the 10^{th} primer pair consisting of a base sequence represented by SEQ ID NO: 19 or 20, the at least one primer pair being capable of detecting Clostridium perfringens;
w) the 11^{th} primer pair consisting of a base sequence represented by SEQ ID NO: 21 or 22, the 11^{th} primer pair being capable of detecting enteroaggregative Escherichia coli (EAEC);
x) at least one primer pair selected from the group consisting of the 12^{th} primer pair consisting of a base sequence represented by SEQ ID NO: 23 or 24, the 13^{th} primer pair consisting of a base sequence represented by SEQ ID NO: 25 or 26, and the 14^{th} primer pair consisting of a base sequence represented by SEQ ID NO: 27 or 28, the at least one primer pair being capable of detecting enterohemorrhagic Escherichia coli (EHEC);
y) the 15^{th} primer pair consisting of a base sequence represented by SEQ ID NO: 29 or 30, the 15^{th} primer pair being capable of detecting enteropathogenic Escherichia coli (EPEC);
z) at least one primer pair selected from the group consisting of the 16^{th} primer pair consisting of a base sequence represented by SEQ ID NO: 31 or 32 and the 17^{th} primer pair consisting of a base sequence represented by SEQ ID NO: 33 or 34, the at least one primer pair being capable of detecting enteroinvasive Escherichia coli (EIEC);
aa) the 18^{th} primer pair consisting of a base sequence represented by SEQ ID NO: 35 or 36, the 18^{th} primer pair being capable of detecting enterotoxigenic Escherichia coli (ETEC);
bb) at least one primer pair selected from the group consisting of the 19^{th} primer pair consisting of a base sequence represented by SEQ ID NO: 37 or 38, the 20^{th} primer pair consisting of a base sequence represented by SEQ ID NO: 39 or 40, and the 21^{st} primer pair consisting of a base sequence represented by SEQ ID NO: 41 or 42, the at least one primer pair being capable of detecting Listeria monocytogenes;
cc) at least one primer pair selected from the group consisting of the 22^{nd} primer pair consisting of a base sequence represented by SEQ ID NO: 43 or 44 and the 23^{rd} primer pair consisting of a base sequence represented by SEQ ID NO: 45 or 46, the at least one primer pair being capable of detecting Salmonella typhimurium;
dd) at least one primer pair selected from the group consisting of the 24^{th} primer pair consisting of a base sequence represented by SEQ ID NO: 47 or 48, the 25^{th} primer pair consisting of a base sequence represented by SEQ ID NO: 49 or 50, the 26^{th} primer pair consisting of a base sequence represented by SEQ ID NO: 51 or 52, the 27^{th} primer pair consisting of a base sequence represented by SEQ ID NO: 53 or 54, and the 28^{th} primer pair consisting of a base sequence represented by SEQ ID NO: 55 or 56, the at least one primer pair being capable of detecting Staphylococcus aureus;
ee) at least one primer pair selected from the group consisting of the 29^{th} primer pair consisting of a base sequence represented by SEQ ID NO: 57 or 58, the 30^{th} primer pair consisting of a base sequence represented by SEQ ID NO: 59 or 60, and the 31^{st} primer pair consisting of a base sequence represented by SEQ ID NO: 61 or 62, the at least one primer pair being capable of detecting Vibrio cholerae;
ff) at least one primer pair selected from the group consisting of the 32^{nd} primer pair consisting of a base sequence represented by SEQ ID NO: 63 or 64, the 33^{rd} primer pair consisting of a base sequence represented by SEQ ID NO: 65 or 66, the 34^{th} primer pair consisting of a base sequence represented by SEQ ID NO: 67 or 68, and the 35^{th} primer pair consisting of a base sequence represented by SEQ ID NO: 69 or 70, the at least one primer pair being capable of detecting Vibrio parahaemolyticus;
gg) at least one primer pair selected from the group consisting of the 36^{th} primer pair consisting of a base sequence represented by SEQ ID NO: 71 or 72 and the 37^{th} primer pair consisting of a base sequence represented by SEQ ID NO: 73 or 74, the at least one primer pair being capable of detecting Vibrio vulnificus; and
hh) at least one primer pair selected from the group consisting of the 38^{th} primer pair consisting of a base sequence represented by SEQ ID NO: 75 or 76 and the 39^{th} primer pair consisting of a base sequence represented by SEQ ID NO: 77 or 78, the at least one primer pair being capable of detecting Yersinia enterocolitica.

8. A composition for detecting food poisoning bacteria, the composition comprising the primer set of claim 1.

9. A next-generation sequencing panel for detecting food poisoning bacteria, the panel comprising the primer set of claim 1.

10. A kit for detecting food poisoning bacteria, the kit comprising the primer set of claim 1.

11. A next-generation sequencing method comprising the steps of:
amplifying a target sequence by using the primer set of claim 1 and a sample;
preparing a library by using the amplified target sequence; and
performing a next-generation sequencing analysis by using the prepared library.

12. A method for detecting food poisoning bacteria, the method comprising the step of performing an amplification reaction by using the primer set of claim 1 and a sample.


SEQUENCE LISTING (78 sequences)

SEQ ID NO: 1 - DNA, 20 bp, Artificial Sequence
  Synthetic primer 1-F
gcgctcttctaaagtctcac

SEQ ID NO: 2 - DNA, 20 bp, Artificial Sequence
  Synthetic primer 1-R
cgaaattagcccagtagcac

SEQ ID NO: 3 - DNA, 21 bp, Artificial Sequence
  Synthetic primer 2-F
gaactgctggtacaacacctg

SEQ ID NO: 4 - DNA, 21 bp, Artificial Sequence
  Synthetic primer 2-R
tctgcactaatgaactgaccg

SEQ ID NO: 5 - DNA, 23 bp, Artificial Sequence
  Synthetic primer 3-F
ccgcttttggacctcaatctcgc

SEQ ID NO: 6 - DNA, 24 bp, Artificial Sequence
  Synthetic primer 3-R
gccttgtgcgcgttctttattgcc

SEQ ID NO: 7 - DNA, 21 bp, Artificial Sequence
  Synthetic primer 4-F
gcaagaattacctttggttga

SEQ ID NO: 8 - DNA, 21 bp, Artificial Sequence
  Synthetic primer 4-R
cctccatttgcttgtattcat

SEQ ID NO: 9 - DNA, 21 bp, Artificial Sequence
  Synthetic primer 5-F
cctacgacttgtttgatttgg

SEQ ID NO: 10 - DNA, 21 bp, Artificial Sequence
  Synthetic primer 5-R
ccacagaatgacctccattag

SEQ ID NO: 11 - DNA, 20 bp, Artificial Sequence
  Synthetic primer 6-F
tggagttgttgtggggcttc

SEQ ID NO: 12 - DNA, 20 bp, Artificial Sequence
  Synthetic primer 6-R
tgatgcgttcttttgcaccc

SEQ ID NO: 13 - DNA, 21 bp, Artificial Sequence
  Synthetic primer 7-F
ttatggcgagggacttggagc

SEQ ID NO: 14 - DNA, 21 bp, Artificial Sequence
  Synthetic primer 7-R
agaataatgcgccgttggacg

SEQ ID NO: 15 - DNA, 21 bp, Artificial Sequence
  Synthetic primer 8-F
atgttactgccgttgatagcg

SEQ ID NO: 16 - DNA, 22 bp, Artificial Sequence
  Synthetic primer 8-R
gctgcataatcccaatcatccc

SEQ ID NO: 17 - DNA, 22 bp, Artificial Sequence
  Synthetic primer 9-F
ggcgttcttctaactcataccc

SEQ ID NO: 18 - DNA, 22 bp, Artificial Sequence
  Synthetic primer 9-R
ctccatcacctaaggactgttc

SEQ ID NO: 19 - DNA, 21 bp, Artificial Sequence
  Synthetic primer 10-F
gccttattgtcttattggtct

SEQ ID NO: 20 - DNA, 21 bp, Artificial Sequence
  Synthetic primer 10-R
gaaaaatggaatagcacctaa

SEQ ID NO: 21 - DNA, 21 bp, Artificial Sequence
  Synthetic primer 11-F
gatgctgacgattctgtatta

SEQ ID NO: 22 - DNA, 21 bp, Artificial Sequence
  Synthetic primer 11-R
ataagtccttctcgattgtgt

SEQ ID NO: 23 - DNA, 21 bp, Artificial Sequence
  Synthetic primer 12-F
gatagatccagaggaagggcg

SEQ ID NO: 24 - DNA, 21 bp, Artificial Sequence
  Synthetic primer 12-R
tacgactgatccctgcaacac

SEQ ID NO: 25 - DNA, 21 bp, Artificial Sequence
  Synthetic primer 13-F
actgtctgaaactgctcctgt

SEQ ID NO: 26 - DNA, 21 bp, Artificial Sequence
  Synthetic primer 13-R
ggttgactctcttcattcacg

SEQ ID NO: 27 - DNA, 23 bp, Artificial Sequence
  Synthetic primer 14-F
ctcaggatgctaaaccagtagag

SEQ ID NO: 28 - DNA, 22 bp, Artificial Sequence
  Synthetic primer 14-R
ccggtacaagcaggattacaac

SEQ ID NO: 29 - DNA, 21 bp, Artificial Sequence
  Synthetic primer 15-F
tagtggattggactcaacgat

SEQ ID NO: 30 - DNA, 21 bp, Artificial Sequence
  Synthetic primer 15-R
tattaacaccgtagcctttcg

SEQ ID NO: 31 - DNA, 23 bp, Artificial Sequence
  Synthetic primer 16-F
acgagtcaacttttagcgaaggg

SEQ ID NO: 32 - DNA, 24 bp, Artificial Sequence
  Synthetic primer 16-R
ctctatttccaatcgcgtcagaac

SEQ ID NO: 33 - DNA, 20 bp, Artificial Sequence
  Synthetic primer 17-F
tcctgcttagatgatggagg

SEQ ID NO: 34 - DNA, 20 bp, Artificial Sequence
  Synthetic primer 17-R
ccaaaaggaagtgtctgctc

SEQ ID NO: 35 - DNA, 21 bp, Artificial Sequence
  Synthetic primer 18-F
tgacggatatgtttccacttc

SEQ ID NO: 36 - DNA, 21 bp, Artificial Sequence
  Synthetic primer 18-R
gtattccacctaacgcagaaa

SEQ ID NO: 37 - DNA, 21 bp, Artificial Sequence
  Synthetic primer 19-F
ctggtgatactctttggggta

SEQ ID NO: 38 - DNA, 22 bp, Artificial Sequence
  Synthetic primer 19-R
agccgttagattcggttgtttc

SEQ ID NO: 39 - DNA, 21 bp, Artificial Sequence
  Synthetic primer 20-F
gtgacgaagtagaagttatcg

SEQ ID NO: 40 - DNA, 21 bp, Artificial Sequence
  Synthetic primer 20-R
agttagtgtgtggagtaatcg

SEQ ID NO: 41 - DNA, 21 bp, Artificial Sequence
  Synthetic primer 21-F
ttgatggtgctgttgcggttc

SEQ ID NO: 42 - DNA, 19 bp, Artificial Sequence
  Synthetic primer 21-R
tgggagttggattgggtgc

SEQ ID NO: 43 - DNA, 21 bp, Artificial Sequence
  Synthetic primer 22-F
cgcactgaatatcgtactgga

SEQ ID NO: 44 - DNA, 20 bp, Artificial Sequence
  Synthetic primer 22-R
cgataatttcaccggcatcg

SEQ ID NO: 45 - DNA, 21 bp, Artificial Sequence
  Synthetic primer 23-F
gctgagtaaccaacaagataa

SEQ ID NO: 46 - DNA, 21 bp, Artificial Sequence
  Synthetic primer 23-R
agtaaacgctgttcataggtc

SEQ ID NO: 47 - DNA, 21 bp, Artificial Sequence
  Synthetic primer 24-F
gcagcttgcttacttactgct

SEQ ID NO: 48 - DNA, 21 bp, Artificial Sequence
  Synthetic primer 24-R
tacctgtaatctcgccatcat

SEQ ID NO: 49 - DNA, 21 bp, Artificial Sequence
  Synthetic primer 25-F
attcattgccctaacgtggac

SEQ ID NO: 50 - DNA, 25 bp, Artificial Sequence
  Synthetic primer 25-R
gctgtaaaaattgatcgtgactctc

SEQ ID NO: 51 - DNA, 20 bp, Artificial Sequence
  Synthetic primer 26-F
gtatggtggtgtaactgagc

SEQ ID NO: 52 - DNA, 20 bp, Artificial Sequence
  Synthetic primer 26-R
ccgtttcataaggcgagttg

SEQ ID NO: 53 - DNA, 21 bp, Artificial Sequence
  Synthetic primer 27-F
ctgctatttttcatccaaaga

SEQ ID NO: 54 - DNA, 21 bp, Artificial Sequence
  Synthetic primer 27-R
ttcttatcagtttgcacttca

SEQ ID NO: 55 - DNA, 20 bp, Artificial Sequence
  Synthetic primer 28-F
tgtcactccacacgaaggta

SEQ ID NO: 56 - DNA, 20 bp, Artificial Sequence
  Synthetic primer 28-R
tgcaaatagcgccttgcttg

SEQ ID NO: 57 - DNA, 21 bp, Artificial Sequence
  Synthetic primer 29-F
gccaagaggacagagtgagta

SEQ ID NO: 58 - DNA, 20 bp, Artificial Sequence
  Synthetic primer 29-R
atgaggactgtatgccccta

SEQ ID NO: 59 - DNA, 21 bp, Artificial Sequence
  Synthetic primer 30-F
taagaccaacagcaaccgccc

SEQ ID NO: 60 - DNA, 23 bp, Artificial Sequence
  Synthetic primer 30-R
actcgactggcgtaaccaaaagg

SEQ ID NO: 61 - DNA, 21 bp, Artificial Sequence
  Synthetic primer 31-F
gtttgtatgtgcgagcgggtg

SEQ ID NO: 62 - DNA, 21 bp, Artificial Sequence
  Synthetic primer 31-R
gtgaatgtcagcgccaccaac

SEQ ID NO: 63 - DNA, 21 bp, Artificial Sequence
  Synthetic primer 32-F
gcactgtctcatttcccagag

SEQ ID NO: 64 - DNA, 21 bp, Artificial Sequence
  Synthetic primer 32-R
cgcttcttggtcagaaaccag

SEQ ID NO: 65 - DNA, 19 bp, Artificial Sequence
  Synthetic primer 33-F
acctgtggcttctgctgtg

SEQ ID NO: 66 - DNA, 21 bp, Artificial Sequence
  Synthetic primer 33-R
ccagttgttgatttgcgggtg

SEQ ID NO: 67 - DNA, 22 bp, Artificial Sequence
  Synthetic primer 34-F
tccatctgtcccttttcctgcc

SEQ ID NO: 68 - DNA, 25 bp, Artificial Sequence
  Synthetic primer 34-R
cagccatttagtacctgacgttgtg

SEQ ID NO: 69 - DNA, 21 bp, Artificial Sequence
  Synthetic primer 35-F
gcgagcgatccttgtttggac

SEQ ID NO: 70 - DNA, 21 bp, Artificial Sequence
  Synthetic primer 35-R
gcggtgagttgctgttgttgg

SEQ ID NO: 71 - DNA, 20 bp, Artificial Sequence
  Synthetic primer 36-F
ctctgcctagatgtttatgg

SEQ ID NO: 72 - DNA, 21 bp, Artificial Sequence
  Synthetic primer 36-R
caataccatttctgtgctaag

SEQ ID NO: 73 - DNA, 21 bp, Artificial Sequence
  Synthetic primer 37-F
agcacatctctattccttctc

SEQ ID NO: 74 - DNA, 20 bp, Artificial Sequence
  Synthetic primer 37-R
tagcgttgcttcttcagtaa

SEQ ID NO: 75 - DNA, 21 bp, Artificial Sequence
  Synthetic primer 38-F
tggggccatctttccgcatta

SEQ ID NO: 76 - DNA, 21 bp, Artificial Sequence
  Synthetic primer 38-R
taccctgcaccaagcatccaa

SEQ ID NO: 77 - DNA, 21 bp, Artificial Sequence
  Synthetic primer 39-F
aacggggcatctggttctctc

SEQ ID NO: 78 - DNA, 21 bp, Artificial Sequence
  Synthetic primer 39-R
tggtggtgtcaggaaagggac